(19) United States
(12) Patent Application Publication
(10) Pub. No.: US 2015/0332016 A1
(43) Pub. Date: Nov. 19, 2015

(54) BRAIN INFORMATION PROCESSING APPARATUS AND BRAIN INFORMATION PROCESSING METHOD

(71) Applicants: ADVANCED TELECOMMUNICATIONS RESEARCH INSTITUTE INTERNATIONAL, Soraku-gun, Kyoto (JP); NIIGATA UNIVERSITY, Niigata-shi, Niigata (JP)

(72) Inventors: Yukiyasu KAMITANI, Soraku-gun (JP); Isao HASEGAWA, Niigata-shi (JP)

(73) Assignees: ADVANCED TELECOMMUNICATIONS RESEARCH INSTITUTE INTERNATIONAL, Soraku-gun, Kyoto (JP); NIIGATA UNIVERSITY, Niigata-shi, Niigata (JP)

(21) Appl. No.: 14/650,961

(22) PCT Filed: Dec. 9, 2013
(86) PCT No.: PCT/JP2013/082957

§ 371 Date: Jun. 10, 2015

(30) Foreign Application Priority Data
Dec. 11, 2012 (JP) 2012-270747

(51) Int. Cl. G06F 19/00; A61B 5/16; A61B 5/00; A61B 5/055

(52) U.S. Cl. CPC G06F 19/3443; A61B 5/055; A61B 5/16; A61B 5/7246

(57) ABSTRACT

Conventionally, it is impossible to detect an object to which a person feels a similar sense based on latent consciousness. Detection of an object to which a person feels a similar sense is realizable by a brain information processing apparatus using high-level brain activity information indicating a latent consciousness. The brain information processing apparatus has stored therein one or more pieces of brain information that includes brain activity information, which is information on a brain activation level, and object information, and includes: an accepting unit that accepts brain activity information; an object information acquisition unit that acquires one or more pieces of object information associated with one or more pieces of brain activity information that is approximate to the brain activity information to the extent of satisfying a predetermined condition; and an output unit that outputs the object information acquired by the object information acquisition unit.

### TECHNICAL FIELD

The present invention relates to a brain information processing apparatus and the like that use information on brain activities.

### BACKGROUND ART

Conventionally, there has been proposed a technique that can deliver data on a content appropriate for a user based on brain waves of the user (see Patent Documents 1 and 2).

Furthermore, there has been proposed a technique that evaluates a design serving as an evaluation target based on an electroencephalogram response, and interactively changes the design to a most appropriate design (see Patent Document 3).

### CITATION LIST

### Patent Documents

[Patent Document 1] JP 2010-218491A (Page 1, FIG. 1 and the like)
[Patent Document 2] JP 2010-220151A (Page 1, FIG. 1 and the like)
[Patent Document 3] JP 2004-342119A (Page 1, FIG. 1 and the like)

### SUMMARY OF INVENTION

### Technical Problem

However, since the conventional apparatuses use brain waves as brain measurement signals, only broad classification of brain activity patterns is possible and classification with respect to individual objects is not possible. Therefore, it is impossible to perform information processing that takes into consideration feelings, potential evaluations, and the like with respect to individual objects.

### Solution to Problem

A brain information processing apparatus according to a first invention is directed to a brain information processing apparatus including:

a brain information storage unit in which one or more pieces of brain information can be stored, the one or more pieces of brain information including brain activity information, which is information on a brain activation level, when a subject is shown an object, and object information, which is information on the object;

an accepting unit configured to accept brain activity information on a brain activation level when, in order that the one or more pieces of brain information to be stored in the brain information storage unit are constructed, an object different from the object shown to the subject is shown to the same subject or a different subject;

an object information acquisition unit configured to acquire, from the brain information storage unit, one or more pieces of object information associated with one or more pieces of brain activity information that is approximate to the brain activity information accepted by the accepting unit to the extent of satisfying a predetermined condition; and

an output unit configured to output the object information acquired by the object information acquisition unit.

According to such a configuration, it is possible to detect, from a brain information database, an object with respect to which a person latently feels a similar sense to that of an object shown to her or him.

Furthermore, a brain information processing apparatus according to a second invention is directed to a brain information processing apparatus including:

a brain information storage unit in which brain information that includes brain activity information, which is information on a brain activation level, when a subject is shown an object can be stored;

an accepting unit configured to accept two or more pieces of brain activity information on a brain activation level when, in order that the one or more pieces of brain information to be stored in the brain information storage unit are constructed, two or more objects different from the object shown to the subject are shown to the same subject or a different subject and two or more pieces of object information, which is information on the two or more objects;

an object information acquisition unit configured to acquire a degree of similarity between the brain activity information of the brain information stored in the brain information storage unit and the two or more pieces of brain activity information accepted by the accepting unit, so as to acquire object information that is associated with the brain activity information having a predetermined degree of similarity; and

an output unit configured to output the object information acquired by the object information acquisition unit.

According to such a configuration, it is possible to detect an object with respect to which a person latently feels the most similar sense to that of given data when a person is shown two or more objects.

Furthermore, a brain information processing apparatus according to a third invention is directed to the brain information processing apparatus according to the first or second invention, wherein the brain information storage unit includes the brain information in association with personal information, which is information on an individual subject,

the accepting unit accepts the brain activity information and personal information associated with the brain activity information, and

the object information acquisition unit acquires, from the brain information associated with the personal information accepted by the accepting unit, one or more pieces of brain activity information, so as to acquire one or more pieces of object information that are associated with the one or more pieces of brain activity information.

According to such a configuration, it is possible to search for an object, taking into consideration individual characteristics.

Furthermore, a brain information processing apparatus according to a forth invention is directed to the brain information processing apparatus according to any one of the first to third inventions, wherein the object information includes one or more pieces of metadata, which is an attribute value of an object,

the object information acquisition unit includes:
an object information determination part for determining two or more pieces of object information associated with two or more pieces of brain activity information that is approximate to brain activity information serving as a comparison target to the extent of satisfying a predetermined condition; and
a static information acquisition part for performing statistical processing on the metadata of the two or more pieces of object information determined by the object information determination part and acquiring statistical information, and

the output unit outputs the statistical information acquired by the static information acquisition part.

According to such a configuration, it is possible to estimate the attribute value of an object serving as a comparison target, based on the attribute value of the object to which the brain is determined to have a similar sense.

Furthermore, a brain information processing apparatus according to a fifth invention is the brain information processing apparatus according to any one of the first to fourth inventions, wherein the brain activity information is information on an activation level of a predetermined partial area of the brain.

According to such a configuration, it is possible to search for an object using information on the brain activity of a predetermined partial area of the brain.

Furthermore, a brain information processing apparatus according to a sixth invention is the brain information processing apparatus according to the fifth invention, wherein the partial area of the brain is a brain area that includes a visual cortex of the brain.

According to such a configuration, it is possible to search for an object to which a person feels a similar sense with respect to a design, using information on the brain activity of the visual cortex of the brain.

Furthermore, a brain information processing apparatus according to a seventh invention is directed to the brain information processing apparatus according to the fifth invention, wherein the partial area of the brain is a brain area that includes the basal ganglia.

According to such a configuration, it is possible to search for an object to which a person feels a similar unconscious compensation, using information on the brain activity of the basal ganglia of the brain.

Furthermore, a brain information processing apparatus according to an eighth invention is directed to the brain information processing apparatus according to the fifth invention, wherein the partial area of the brain is a brain area that includes the orbitofrontal cortex.

According to such a configuration, it is possible to search for an object to which a person feels a similar unconscious joy or preference, using information on the brain activity of the orbitofrontal cortex of the brain.

Furthermore, a brain information processing apparatus according to a ninth invention is directed to the brain information processing apparatus according to any one of the first to eighth inventions, further including:

an image storage unit in which two or more images that show brain activation levels when a subject is shown two or more objects can be stored;

a feature vector acquisition unit configured to acquire two or more feature vectors that have, as elements, values relating to a change as compared with a baseline of pixel values of two or more pixels constituting each of the two or more images; and

a brain activity information acquisition unit configured to acquire a degree of similarity between the two or more feature vectors, acquire a brain expression similarity matrix, which is a symmetric matrix having the degree of similarity as an element, and accumulate the brain expression similarity matrix in the brain information storage unit.

According to such a configuration, it is possible to automatically construct a brain information database.

### Advantageous Effects of Invention

According to the brain information processing apparatus of the present invention, it is possible to detect an object to which a person feels a similar sense, using information on a detailed brain activity pattern that indicates a latent consciousness.

### BRIEF DESCRIPTION OF DRAWINGS

FIG. 1 is a block diagram showing a brain information processing apparatus 1 according to Embodiment 1.

FIG. 2 is a flowchart illustrating the operation in which the brain information processing apparatus 1 outputs object information according to the embodiment.

FIG. 3 is a flowchart illustrating the operation in which the brain information processing apparatus 1 accumulates brain activity information according to the embodiment.

FIG. 4 is a flowchart illustrating processing for generating a brain expression similarity matrix according to the embodiment.

FIG. 5 is a schematic diagram showing an fMRI device 5 according to the embodiment.

FIG. 6 is a diagram showing information on subjects according to the embodiment.

FIG. 7 is a diagram illustrating processing for constructing a feature vector from an image according to the embodiment.

FIG. 8 is a diagram illustrating ideas of the operations of a feature vector acquisition unit 106 and a brain activity information acquisition unit 107 according to the embodiment.

FIG. 9 is a diagram showing an output example of an RSM according to the embodiment.

FIG. 10 is a diagram showing examples of images that are stored in an image storage unit 101 according to the embodiment.

FIG. 11 is a diagram showing RSMs that correspond to the areas of a subject TH according to the embodiment.

FIG. 12 is a diagram showing brain expression similarity matrices (RSM) according to the embodiment.

FIG. 13 is a diagram showing brain expression similarity matrices (RSM) according to the embodiment.

FIG. 14 is a diagram showing output examples of the brain activity information according to the embodiment.

FIG. 15 is a block diagram of a brain information processing apparatus 2 according to Embodiment 2.

FIG. 16 is a flowchart illustrating the operation of the brain information processing apparatus 2 according to the embodiment.

FIG. 17 is a diagram showing output examples of the brain activity information according to the embodiment.

FIG. 18 is a diagram showing output examples of the brain activity information according to the embodiment.

FIG. 19 is a diagram showing output examples of the brain activity information according to the embodiment.

FIG. 20 is an overview diagram showing a computer system according to the embodiment.

FIG. 21 is a block diagram showing the computer system according to the embodiment.

### DESCRIPTION OF EMBODIMENTS

Hereinafter, embodiments of a brain information processing apparatus and the like will be described with reference to the drawings. Note that in the embodiments, constituent components with the same reference numeral perform the same operation, and thus redundant descriptions thereof will sometimes be omitted.

### Embodiment 1

The present embodiment will describe a brain information processing apparatus in which one or more pieces of brain information, which includes brain activity information on the brain activation level when a subject is shown an object and object information on the object, are compiled into a database, and that searches the database using the brain activity information when a subject is shown a particular object, acquires object information that is associated with similar brain activity information, and outputs the acquired object information.

Furthermore, the present embodiment will describe the brain information processing apparatus in which brain information is associated with information on a subject, and that determines similar brain activity information among the brain information associated with received information on a subject, acquires object information that is associated with the brain activity information, and outputs the acquired object information.

Furthermore, the present embodiment will describe a method for constructing brain activity information.

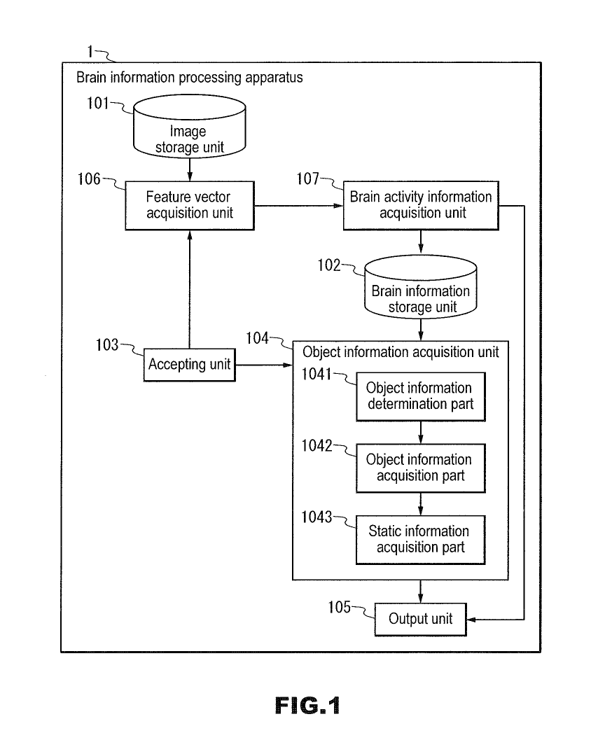

FIG. 1 is a block diagram showing a brain information processing apparatus 1 according to the present embodiment. The brain information processing apparatus 1 is provided with an image storage unit 101, a brain information storage unit 102, an accepting unit 103, an object information acquisition unit 104, an output unit 105, a feature vector acquisition unit 106, and a brain activity information acquisition unit 107.

Furthermore, the object information acquisition unit 104 may include an object information determination part 1041, an object information acquisition part 1042, and a static information acquisition part 1043.

In the image storage unit 101, two or more images may be stored. Each of the two or more images is an image showing the brain activation level when a subject is shown an object. It is preferable that the image be a brain image acquired by, for example, fMRI measurement. Note that the brain image acquired by fMRI measurement is referred to also as an fMRI brain activity pattern. Furthermore, the image may also be a brain image measured using, for example, an NIRS brain measurement device, a PET, or the like. Note that the image may also be a whole-brain image, which shows the whole brain, or an image of a part of the brain. A part of the brain may be, for example, a visual cortex, the basal ganglia, the orbitofrontal cortex, or the like. The visual cortex may be either or both of the lower visual cortex or/and the higher visual cortex.

In the brain information storage unit 102, one or more pieces of brain information may be stored. The brain information includes brain activity information and object information. The brain activity information is information on the brain activation level when a subject is shown an object. The brain activity information is, for example, information using a change in the intensity of an fMRI signal. Note that information using a change in the intensity of an fMRI signal is, for example, information using an amount of change from a baseline value for the intensity of an fMRI signal. The brain activity information is, for example, a feature vector that includes, as elements, values relating to a change as compared with the baseline of pixel values of two or more pixels constituting a brain image acquired by fMRI measurement. Furthermore, the brain activity information is, for example, a brain expression similarity matrix, which will be described later. It is preferable that the brain activity information be information on the activation level of a predetermined partial area of the brain. A partial area of the brain may be, for example, a visual cortex, the basal ganglia, the orbitofrontal cortex, or the like. The visual cortex may be either or both of the lower visual cortex or/and the higher visual cortex. Note that the brain activity information may also be information on the activation level of the whole brain. Furthermore, the brain expression similarity matrix is information that corresponds to two or more pieces of brain activity information.

Furthermore, the brain activity information may also be information obtained from an image acquired by NIRS, brain wave information, or the like. The information obtained from an image acquired by NIRS is, for example, a feature vector that has, as elements, differences between pixel values of pixels of the image and the baseline pixel value.

Furthermore, the object information is information on an object. The object may be a physical object or an image. Any object is applicable as long as it is recognizable by a human. Furthermore, the object information is, for example, the picture of the object, the name of the object, the object itself, the ID of the object, or the like. Furthermore, it is preferable that the object information have one or more pieces of metadata. The metadata is an attribute value of the object, and category or sales of the object, evaluations of a questionnaire about the object, and the like.

Furthermore, it is preferable that the brain information storage unit 102 include brain information in association with personal information, which is information on an individual subject. The personal information is, for example, a personal identifier for identifying a subject, an attribute value of a subject, or the like. The attribute value of a subject is, for example, age, sex, nationality, job, preference, and the like of the subject.

The accepting unit 103 accepts various types of instructions, information and the like. The accepting unit 103 accepts, for example, brain activity information. Furthermore, the accepting unit 103 may also accept brain activity information and personal information that is associated with the brain activity information. Furthermore, the accepting unit 103 may also accept object information. Furthermore, the accepting unit 103 may also accept an instruction to output brain activity information. Note that the brain activity information that is accepted by the accepting unit 103 is information on the brain activation level when, in order that one or more pieces of brain information to be stored in the brain information storage unit 102 are constructed, an object that is different from the object shown to a subject is shown to the same or a different subject.

Here, “accept” refers to an idea that includes accepting information input from an input device such as a keyboard, a mouse, or a touch panel, reception of information transmitted via a wired or wireless communication line, accepting information read out from a recording medium such as an optical disk, a magnetic disc, or a semiconductor memory, and the like.

Any means such as a means using a keyboard, a mouse, or a menu screen may be used for inputting brain activity information. The accepting unit 103 may be realized by a device driver for the input means such as a keyboard, control software for a menu screen, or the like.

The object information acquisition unit 104 acquires, from the brain information storage unit 102, one or more pieces of object information associated with one or more pieces of brain activity information that is approximate to the brain activity information accepted by the accepting unit 103 to the extent of satisfying a predetermined condition.

The object information acquisition unit 104 may acquire one or more pieces of brain activity information from among the brain information associated with the personal information accepted by the accepting unit 103, and acquire one or more pieces of object information associated with the one or more pieces of brain activity information.

If the brain activity information is a brain expression similarity matrix, the object information acquisition unit 104 may acquire one or more pieces of object information that corresponds to the value indicating the approximation of the values of elements of the matrix that corresponds to the object information accepted by the accepting unit 103.

The object information determination part 1041 constituting the object information acquisition unit 104 determines one or two or more pieces of object information associated with one or two or more pieces of brain activity information that is approximate to the brain activity information serving as a comparison target to the extent of satisfying a predetermined condition. Note here that the brain activity information serving as a comparison target is the brain activity information accepted by the accepting unit 103. The object information determination part 1041 determines, for example, N (N is 10, for example) pieces of object information associated with N pieces of brain activity information in the descending order of the degree of similarly to the brain activity information serving as a comparison target.

The object information acquisition part 1042 acquires a part or the entire of the object information determined by the object information determination part 1041. Here, the object information acquisition part 1042 acquires a part or the entire of the object information from the brain information storage unit 102.

The static information acquisition part 1043 performs statistical processing on metadata of the two or more pieces of object information determined by the object information determination part 1041, and acquires statistical information. The statistical processing is, for example, processing for acquiring the average value or the central value, or the like. The statistical information is, for example, the average value, the central value, or the like. Note that the content of the statistical processing is not essential. Furthermore, if the metadata is, for example, an evaluation value of a questionnaire with respect to the object, the static information acquisition part 1043 acquires, for example, the average value of two or more evaluation values included in the two or more pieces of object information. Note that the object information acquisition unit 104 may not necessarily be provided with the static information acquisition part 1043.

The output unit 105 outputs the one or more pieces of object information acquired by the object information acquisition unit 104. Furthermore, the output unit 105 may output the brain activity information acquired by the brain activity information acquisition unit 107, which will be described later. The output unit 105 may output the statistical information acquired by the static information acquisition part 1043. The brain activity information is, for example, a brain expression similarity matrix (RSM), which will be described later. Here, “output” refers to an idea including display on a display screen, projection using a projector, printing by a printer, output of a sound, transmission to an external apparatus, accumulation into a recording medium, delivery of a processing result to another processing apparatus or another program, and the like.

The feature vector acquisition unit 106 acquires two or more feature vectors that have, as elements, values relating to a change as compared with the baseline of pixel values of two or more pixels constituting each of two or more images. Note that the baseline of pixel values of two or more pixels is assumed to be held in advance by the feature vector acquisition unit 106. Furthermore, the feature vector is a one-dimensional vector.

The brain activity information acquisition unit 107 acquires the degree of similarity between the two or more feature vectors, and acquires a brain expression similarity matrix (representational similarity matrix (RSM)), which is a symmetric matrix that has degrees of similarity as elements. This brain expression similarity matrix is an example of the brain activity information. Then, the brain activity information acquisition unit 107 accumulates this brain expression similarity matrix into the brain information storage unit 102. Examples of the brain expression similarity matrix will be described later. Note that the degree of similarity is, for example, a distance between vectors, a value obtained by normalizing the distance between vectors, or the like.

Furthermore, the brain activity information acquisition unit 107 may acquire feature vectors acquired by the feature vector acquisition unit 106 as brain activity information, and may accumulate the feature vectors into the brain information storage unit 102.

The image storage unit 101 and the brain information storage unit 102 are preferably nonvolatile recording media, but are also realizable by volatile recording media. The process in which an image or the like is stored in the image storage unit 101 or the like is not essential. For example, an image or the like may be stored in the image storage unit 101 or the like via a recording medium, an image transmitted via a communication line or the like may be stored in the image storage unit 101 or the like, or an image or the like that was input via an input device may be stored in the image storage unit 101 or the like.

The object information acquisition unit 104, the feature vector acquisition unit 106, and the brain activity information acquisition unit 107 may ordinarily be realized by an MPU, a memory, or the like. The processing procedures of the object information acquisition unit 104 and the like are ordinarily realized by software that is stored in a recording medium such as a ROM. However, the processing procedures may also be realized by hardware (dedicated circuits).

It is conceivable that the output unit 105 includes or does not include an output device such as a display screen or a speaker. The output unit 105 may be realized by driver software for the output device, the output device and the driver software of the output device, or the like.

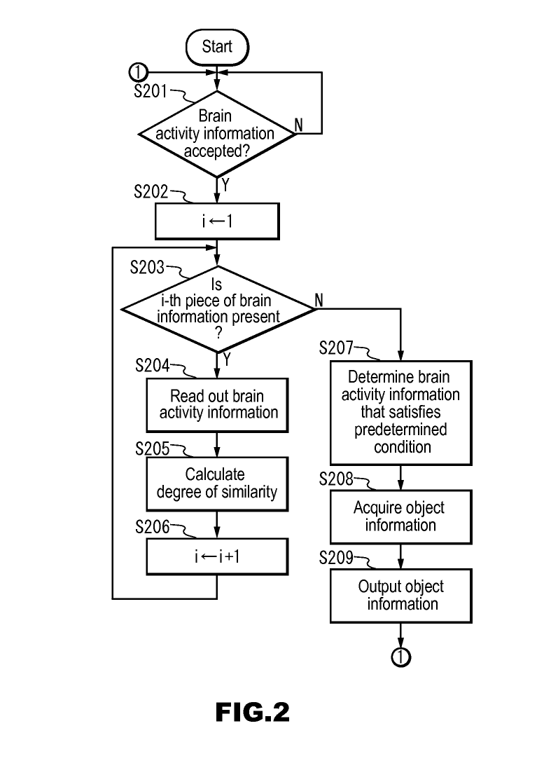

The following will describe the operation in which the brain information processing apparatus 1 outputs object information with reference to the flowchart of FIG. 2.

(Step S201) The accepting unit 103 determines whether or not it has accepted brain activity information. If the accepting unit 103 has accepted brain activity information, the procedure advances to step S202, whereas if the accepting unit 103 has not accepted brain activity information, the procedure returns to step S201.

(Step S202) The object information acquisition unit 104 substitutes 1 for a counter i.

(Step S203) The object information acquisition unit 104 determines whether or not the i-th piece of brain information is present in the brain information storage unit 102. If the i-th piece of brain information is present, the procedure advances to step S204, whereas if the i-th piece of brain information is not present, the procedure advances to step S207.

(Step S204) The object information acquisition unit 104 reads out brain activity information included in the i-th piece of brain information from the brain information storage unit 102.

(Step S205) The object information acquisition unit 104 calculates the degree of similarity between the brain activity information accepted in step S201 and the i-th piece of brain activity information read out in step S204. Then, the object information acquisition unit 104 temporarily accumulates, in a recording medium (not shown), the degree of similarity in association with the i-th piece of brain activity information or information for identifying the i-th piece of brain activity information. Note that the technique for calculating the degree of similarity between vectors is well known, and thus a detailed description thereof is omitted. Note that any method for expressing the degree of similarity may be used. That is, for example, the maximum value of the degree of similarity may be defined as 1 or 100.

(Step S206) The object information acquisition unit 104 increments the counter i by 1. The procedure returns to step S203.

(Step S207) The object information acquisition unit 104 determines, using the information temporarily accumulated in step S205, one or more pieces of brain activity information that satisfies a predetermined condition. Ordinarily, the object information acquisition unit 104 acquires the brain activity information having the largest degree of similarity. The object information acquisition unit 104 may acquire one or more pieces of brain activity information having the degree of similarity that is larger than a threshold.

(Step S208) The object information acquisition unit 104 acquires, from the brain information storage unit 102, one or more pieces of object information that are paired with the one or more pieces of brain activity information determined in step S207. Note that the object information acquisition unit 104 may also acquire two or more pieces of object information in the descending order of the degree of similarity.

(Step S209) The output unit 105 outputs the one or more pieces of object information acquired in step S208.

Note that in the flowchart of FIG. 2, the processing ends due to power off or an interruption at the end of the processing.

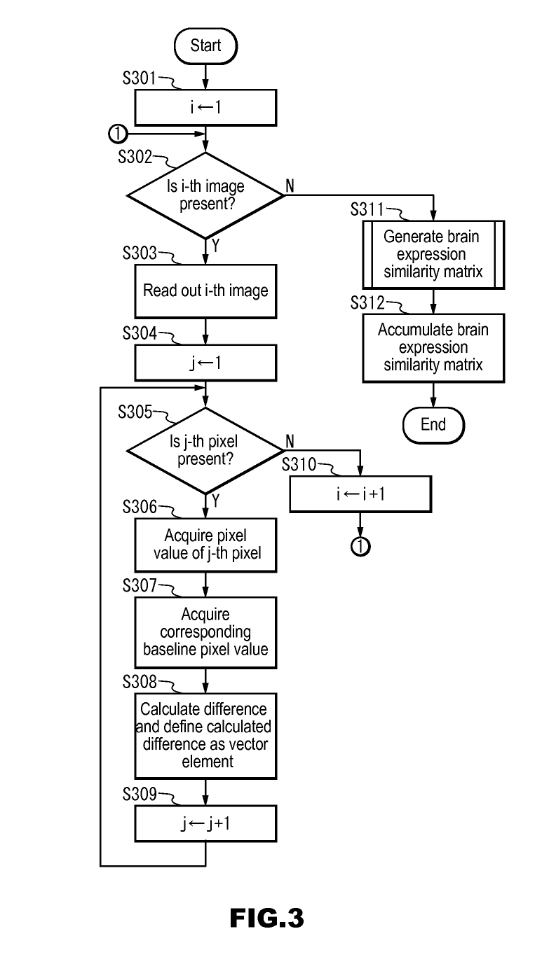

The following will describe the operation in which the brain information processing apparatus 1 accumulates brain activity information with reference to the flowchart of FIG. 3.

(Step S301) The feature vector acquisition unit 106 substitutes 1 for a counter i.

(Step S302) The feature vector acquisition unit 106 determines whether or not the i-th image is present in the image storage unit 101. If the i-th image is present, the procedure advances to step S303, whereas if the i-th image is not present, the procedure advances to step S311.

(Step S303) The feature vector acquisition unit 106 reads out the i-th image from the image storage unit 101.

(Step S304) The feature vector acquisition unit 106 substitutes 1 for a counter j.

(Step S305) The feature vector acquisition unit 106 determines whether or not the j-th pixel is present in the i-th image or a predetermined area of the i-th image. If the j-th pixel is present, the procedure advances to step S306, whereas if the j-th pixel is not present, the procedure advances to step S310. Note that the predetermined area of the i-th image is, for example, a partial area of a brain image and for example, a visual cortex, the basal ganglia, the orbitofrontal cortex, or the like.

(Step S306) The feature vector acquisition unit 106 acquires the pixel value of the j-th pixel.

(Step S307) The feature vector acquisition unit 106 acquires the baseline pixel value that corresponds to the j-th pixel. Note that the baseline image is assumed to be stored in, for example, the image storage unit 101 or the feature vector acquisition unit 106.

(Step S308) The feature vector acquisition unit 106 calculates a difference between the pixel value of the j-th pixel and the baseline pixel value, and accumulates the calculated difference as the j-th element of the vectors. Note that an area in which the vectors are stored is assumed to be ensured.

(Step S309) The feature vector acquisition unit 106 increments the counter j by 1. The procedure returns to step S305.

(Step S310) The feature vector acquisition unit 106 increments the counter i by 1. The procedure returns to step S302.

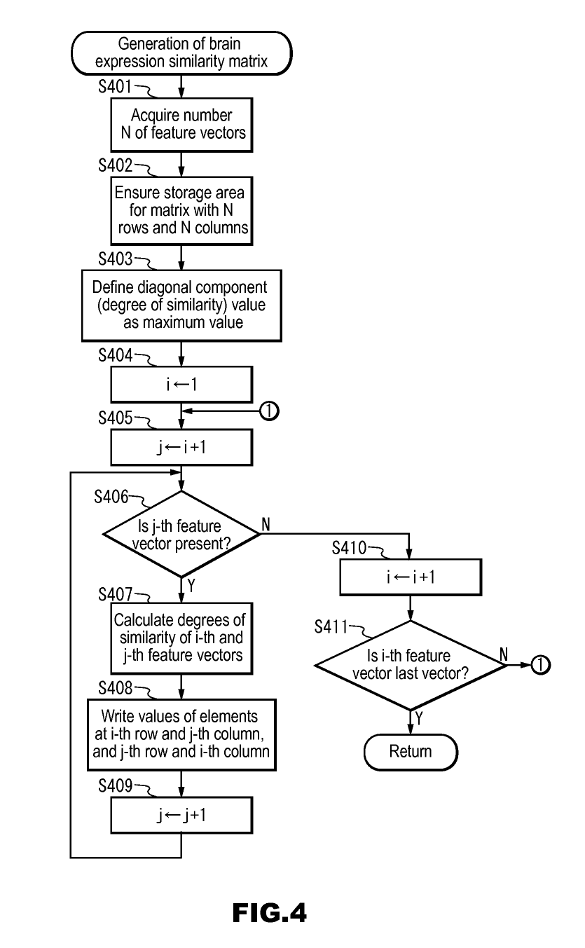

(Step S311) The brain activity information acquisition unit 107 generates a brain expression similarity matrix using two or more vectors. This processing for generating a brain expression similarity matrix will be described with reference to the flowchart of FIG. 4.

(Step S312) The brain activity information acquisition unit 107 accumulates the brain expression similarity matrix generated in step S311 in the brain information storage unit 102. The processing ends.

Note that in step S312 of the flowchart of FIG. 3, the brain activity information acquisition unit 107 preferably accumulates, in the brain information storage unit 102, the brain expression similarity matrix in which the row number and the column number thereof are associated with the object information. Note that the object information that is associated with the row number is information for specifying an image that serves as a basis for the feature vector corresponding to the row number. The information for specifying an image refers to an image itself, the object name or ID for specifying an object captured on the image, or the like.

Furthermore, in step S312 of the flowchart of FIG. 3, instead of or in addition to the brain activity information acquisition unit 107 accumulating the brain expression similarity matrix, the output unit 105 may output the brain expression similarity matrix.

Furthermore, in step S311 of the flowchart of FIG. 3, the brain activity information acquisition unit 107 may accumulate, in the brain information storage unit 102, feature vectors that correspond to each image in association with the object information for specifying the image that serves as a basis for the feature vectors.

The following will describe the processing for generating a brain expression similarity matrix in step S311 with reference to the flowchart of FIG. 4.

(Step S401) The brain activity information acquisition unit 107 acquires a number N of vectors acquired by the feature vector acquisition unit 106.

(Step S402) The brain activity information acquisition unit 107 ensures a storage area for a matrix with N rows and N columns.

(Step S403) The brain activity information acquisition unit 107 defines the value of the diagonal component (degree of similarity) of the matrix with N rows and N columns, as the maximum value. In other words, the brain activity information acquisition unit 107 records the maximum degree of similarity (for example, 1) in the area of the diagonal component of the matrix with N rows and N columns.

(Step S404) The brain activity information acquisition unit 107 substitutes 1 for the counter i.

(Step S405) The brain activity information acquisition unit 107 substitutes “i+1” for the counter j.

(Step S406) The brain activity information acquisition unit 107 determines whether or not the j-th feature vector is present. If the j-th feature vector is present, the procedure advances to step S407, whereas if the j-th feature vector is not present, the procedure advances to step S410.

(Step S407) The brain activity information acquisition unit 107 calculates the degree of similarity between the i-th feature vector and the j-th feature vector.

(Step S408) The brain activity information acquisition unit 107 writes the degree of similarity obtained in step S407 as the value of the element at the i-th row and the j-th column, and the element at the j-th row and the i-th column, among the matrix with N rows and N columns.

(Step S409) The brain activity information acquisition unit 107 increments the counter j by 1. The procedure returns to step S406.

(Step S410) The brain activity information acquisition unit 107 increments the counter i by 1.

(Step S411) The brain activity information acquisition unit 107 determines whether or not the i-th feature vector is the last vector. If the i-th feature vector is the last vector, the procedure returns to the superordinate processing, whereas if the i-th feature vector is not the last vector, the procedure advances to step S405.

Hereinafter, specific operations of the present embodiment will be described.

### Specific Example 1

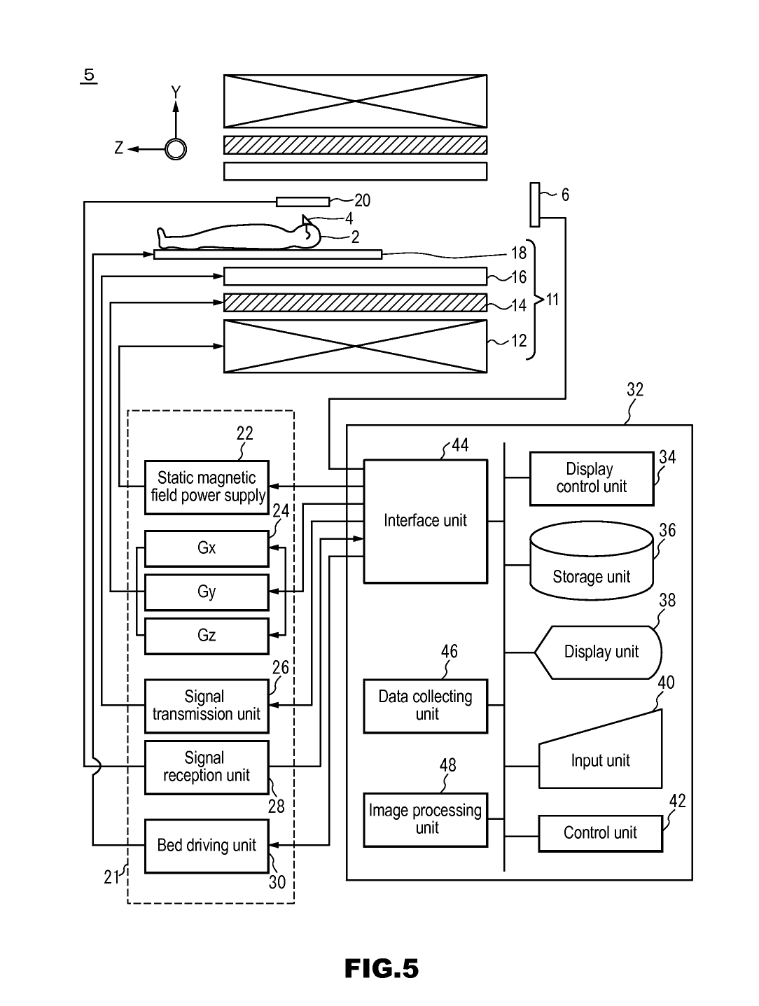

Specific Example 1 is a specific example of an apparatus for acquiring brain activity information for use by the brain information processing apparatus 1. A schematic diagram showing the overall configuration of the apparatus for acquiring brain activity information for use by the brain information processing apparatus 1 is shown in FIG. 5. FIG. 5 is a schematic diagram of an fMRI device 5.

As shown in FIG. 5, the fMRI device 5 is provided with a magnetic field application mechanism 11 for applying a controlled magnetic field to an area of interest of a subject 2 to irradiate the area of interest with RF waves; a receiving coil 20 that receives response waves (NMR signals) from this subject 2 and outputs analog signals; a driving unit 21 that controls a magnetic field that is applied to this subject 2 and controls transmission and reception of RF waves; and a data processing unit 32 that sets a control sequence of the driving unit 21 and processes various types of data signals to generate an image.

Note here that the central axis of a cylindrically-shaped bore in which the subject 2 is placed is the Z-axis, and the X-axis is defined as the axis in the horizontal direction that is orthogonal to the Z-axis, and the Y-axis is defined as the axis in the vertical direction that is orthogonal to the Z-axis.

Since the fMRI device 5 has such a configuration, nuclear spins of the atomic nuclei constituting the subject 2 are aligned in the magnetic field direction (Z-axis) due to a static magnetic field applied by the magnetic field application mechanism 11, and perform precession movement in this magnetic field direction at the Larmor frequency that is unique to this atomic nuclei.

If the subject 2 is irradiated with an RF pulse having the same frequency as that of the Larmor frequency, the atoms resonate, absorb an energy, and are excited, that is, a nuclear magnetic resonance phenomenon (NMR phenomenon; Nuclear Magnetic Resonance) occurs. If the RF pulse radiation is stopped after the resonance of the atoms, the atoms output, during the relaxation process of emitting the energy and returning to the original static state, electromagnetic waves (NMR signals) having the same frequency as that of the Larmor frequency.

The output NMR signals are received by the receiving coil 20 as response waves from the subject 2, and an area of interest of the subject 2 is imaged in the data processing unit 32. This image is the image stored in the image storage unit 101.

The magnetic field application mechanism 11 is provided with a static magnetic field generation coil 12, an inclined magnetic field generation coil 14, an RF radiation unit 16, and a bed 18 on which the subject 2 is placed in the bore.

The subject 2 is not particularly limited, but can see a screen displayed on a display screen 6 that is installed perpendicular to the Z-axis using, for example, a prism glasses 4. An image on this display screen 6 gives the subject 2 a visual stimulus.

Note that the visual stimulus to the subject 2 may be configured to be given by an object image that is projected in front of the eyes of the subject 2 by a projector.

The driving unit 21 is provided with a static magnetic field power supply 22, an inclined magnetic field power supply 24, a signal transmission unit 26, a signal reception unit 28, and a bed driving unit 30 that moves the bed 18 to an arbitrary position in the Z-axis direction.

The data processing unit 32 is provided with: an input unit 40 that accepts inputs of various types of operations or information from an operator (not shown); a display unit 38 that displays, on the screen, various types of images and information relating to an area of interest of the subject 2; a storage unit 36 in which programs for executing various types of processing, control parameters, image data (such as a three-dimensional model image), and other types of electronic data are stored; a control unit 42 that controls operations of various functional units, that is, for example, generates a control sequence for driving the driving unit 21; an interface unit 44 that executes transmission and reception of various types of signals to and from the driving unit 21; a data collecting unit 46 that collects data constituted by a group of NMR signals derived from an area of interest; and an image processing unit 48 that forms an image based on the data on the NMR signals.

Furthermore, the data processing unit 32 is a dedicated computer, or a general-purpose computer that executes the functions for operating the functional units, including a processing unit that performs designated calculation or data processing, or generates a control sequence, based on a program installed in the storage unit 36.

The static magnetic field generation coil 12 generates a static magnetic field in the Z-axis in the bore, by a current supplied from the static magnetic field power supply 22 flowing through the spiral coil wound about the Z-axis to generate an induction magnetic field. An area that is formed in the bore and has a highly uniform static magnetic field will be set as the area of interest of the subject 2. More specifically, the static magnetic field coil 12 is constituted by, for example, four air core coils, creates a uniform magnetic field therein using the combination of the four air core coils, and gives orientation to the spins of predetermined atomic nuclei, more specifically, hydrogen atomic nuclei inside the body of the subject 2.

The inclined magnetic field generation coil 14 is constituted by an X-coil, a Y-coils and a Z-coil (illustrations thereof are omitted), and is provided on the inner peripheral surface of the cylindrically-shaped static magnetic field generation coil 12.

These X-coil, Y-coil, and Z-coil superimpose the inclined magnetic field onto the uniform magnetic field in the bore while switching the X-axis direction, the Y-axis direction, and the Z-axis direction in order, and gives the strength inclination to the static magnetic field. The Z-coil inclines the magnetic field strength in the Z-direction to restrict the resonance surface at the time of excitation, and the Y-coil gives an inclination in a short time period immediately after application of the magnetic field in the Z-direction so as to add phase modulation that is proportional to the Y-coordinate to a detected signal (phase encoding), and then the X-coil gives an inclination at the time of data collection so as to add frequency modulation that is proportional to the X-coordinate to a detected signal (frequency encoding).

The switching of the inclined magnetic field that is to be superimposed is realized by different pulse signals being output to the X-coil, the Y-coil, and the Z-coil from the transmission unit 24 in accordance with the control sequence. Accordingly, it is possible to specify the position of the subject 2 at which the NMR phenomenon occurs, and to give the positional information on the three-dimensional coordinates that are needed for forming the image of the subject 2.

The RF radiation unit 16 irradiates the area of interest of the subject 2 with a RF (Radio Frequency) pulse based on a high frequency signal transmitted from the signal transmission unit 33 in accordance with the control sequence.

Note that in FIG. 5, the RF radiation unit 16 is included in the magnetic field application mechanism 11, but may be provided on the bed 18 or formed into one piece with the receiving coil 20.

The receiving coil 20 detects response waves (NMR signals) from the subject 2, and is arranged close to the subject 2 in order to achieve the detection of the NMR signals with high sensitivity.

Here, in the receiving coil 20, a weak current occurs based on electromagnetic induction when electromagnetic waves of the NMR signal come across its coil bare wire. This weak current is amplified in the signal reception unit 28, is converted into a digital signal from the analog signal, and is transmitted to the data processing unit 32.

That is, a high frequency electromagnetic field of a resonance frequency is applied, via the RF radiation unit 16, to the subject 2 in the state in which a Z-axis inclined electromagnetic field is applied to the static magnetic field. Accordingly, predetermined atomic nuclei that have a resonance condition of the magnetic field strength, that is, for example, hydrogen atomic nuclei are selectively excited to start resonating. The atomic nuclei in this section that satisfy the resonance condition (for example, a layer with a predetermined thickness of the subject 2) are excited, and the spins thereof rotate all together. If the exciting pulse is stopped, electromagnetic waves emitted by the rotation of the spins induce a signal of the receiving coil 20, and this signal is detected for a while. With this signal, the structure including the predetermined atoms in the body of the subject 2 is monitored. Then, X and Y inclined electromagnetic fields are applied in order to recognize the position at which the signal is generated, and the signal is detected.

The image processing unit 48 measures a detected signal while repeatedly applying an excitation signal based on the data constructed in the storage unit 36, reduces the resonance frequency to the X-coordinate by the first Fourier transform computation, restores the resonance frequency to the Y-coordinate by the second Fourier transform, thereby acquiring an image, and displays the corresponding image on the display unit 38.

### Specific Example 2

In Specific Example 2, an experiment using the brain information processing apparatus 1 will be described. Furthermore, in Embodiment 2, an image showing the brain activation level that is stored in the image storage unit 101 is a whole-brain image. Also, brain activity information constituting brain information that is stored in the brain information storage unit 102 is information on the activation level of the whole brain when a subject is shown an object.

It is here assumed that a large number of stimulus images are stored in a storing means (not shown). The stimulus images are images that are shown to a subject, and here encompass a large number of character images, a large number of drink can images, a large number of car images, a large number of building images, a large number of aromatic container images, or a large number of cosmetics images.

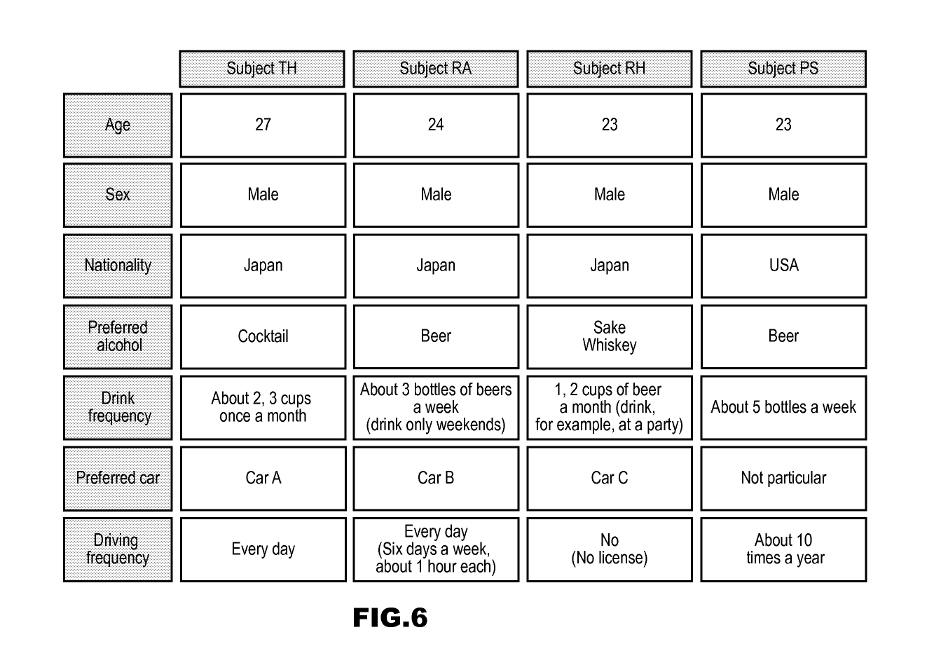

The stimulus images were displayed using the display screen or the projector and shown to four subjects. Then, the brain activities of the four subjects when being shown the stimulus images were measured using the fMRI device 5. The four subjects are a subject TH, a subject RA, a subject RH, and a subject PS. Information on the four subjects is shown in FIG. 6.

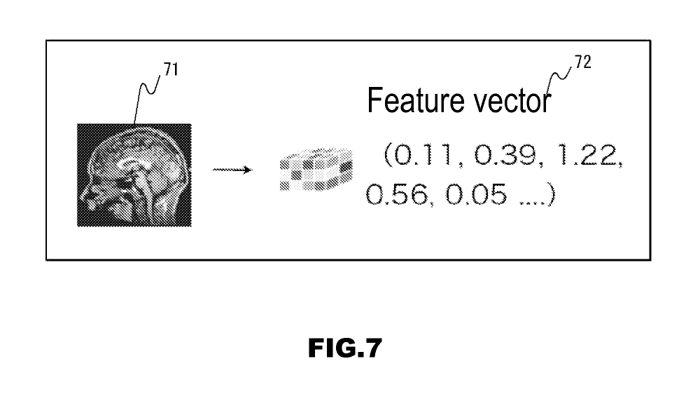

Then, a large number of images that were acquired by the fMRI device 5 when the subjects are shown the stimulus images are stored in the image storage unit 101. An example of the images stored in the image storage unit 101 is shown by the reference numeral 71 of FIG. 7. Note that the reference numeral 71 denotes here an fMRI brain activity pattern.

It is assumed that, in such a situation, a user has input an instruction to output brain activity information into the brain information processing apparatus 1. Then, the accepting unit 103 accepts the instruction to output brain activity information.

Then, the feature vector acquisition unit 106 acquires the images stored in the image storage unit 101. Then, the feature vector acquisition unit 106 acquires two or more pixel values of each image.

Then, the feature vector acquisition unit 106 acquires a baseline of the pixel values of pixels of the brain image. Note that the brain image that serves as a basis of the baseline is assumed to be held in advance by, for example, the feature vector acquisition unit 106.

Then, the feature vector acquisition unit 106 acquires two or more feature vectors (reference numeral 72 of FIG. 7) that have differences between the pixel values and the baseline pixel value of each image, as elements. Note that the feature vectors are generated for each image. Furthermore, the difference from the baseline pixel value is, here, information indicating a change in the intensity of an fMRI signal (% signal change) when the subject is shown the image.

Then, the brain activity information acquisition unit 107 acquires the degree of similarity between the two or more feature vectors. Here, the degree of similarity is a value indicating the correlation between the two feature vectors, that is, a value obtained by normalizing the distance between the two feature vectors from 1 to −1. Note that “1” is the largest degree of similarity and “−1” is the smallest degree of similarity. Furthermore, the degree of similarity between feature vectors may also be referred to as correlation.

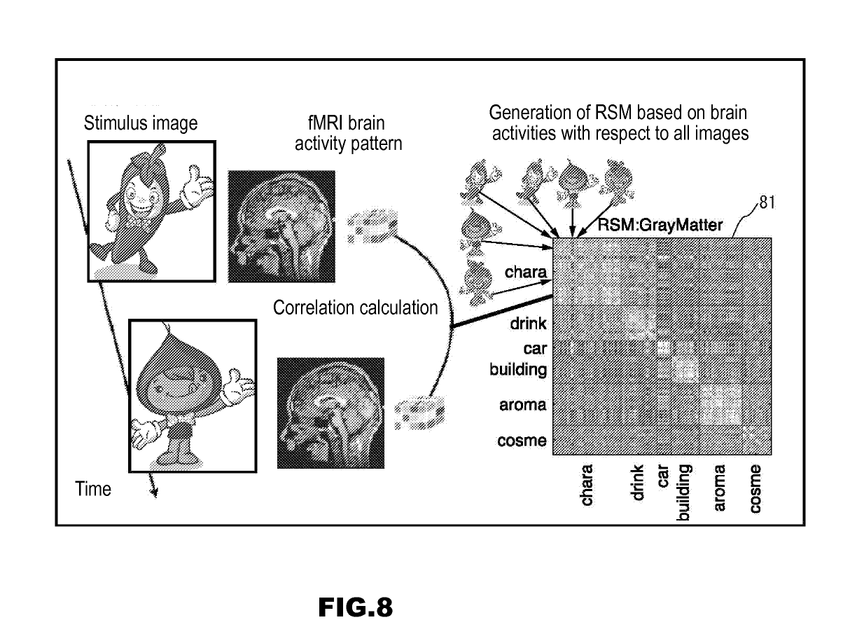

Then, the brain activity information acquisition unit 107 acquires a brain expression similarity matrix (RSM), which is a symmetric matrix that has degrees of similarity as elements. For example, the brain activity information acquisition unit 107 is assumed to acquire the RSM shown by the reference numeral 81 of FIG. 8. Note that FIG. 8 is a diagram illustrating an idea of operations of the feature vector acquisition unit 106 and the brain activity information acquisition unit 107. In FIG. 8, the subject was shown the stimulus images (for example, the character images of FIG. 8, or the like) in order. Note that images shown to the subject are a large number of character images, drink container images, a large number of car images, a large number of building images, a large number of aromatic container images, and a large number of cosmetics images.

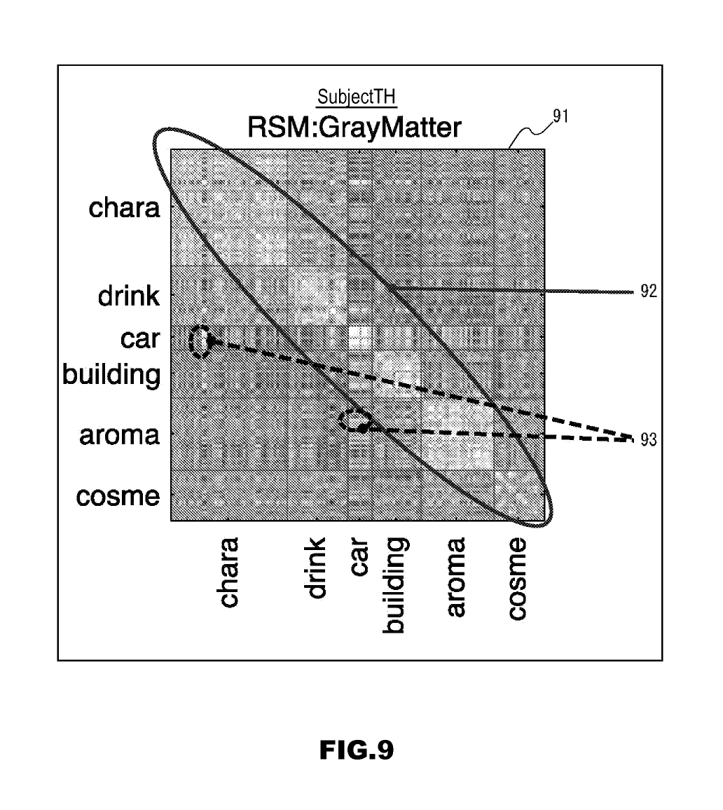

Then, the output unit 105 outputs the acquired RSM. An output example of the RSM is shown in FIG. 9. The reference numeral 91 of FIG. 9 denotes the RSM acquired based on the brain activity information of the subject TH. In FIG. 9, on the axes, “chara” indicates characters, “drink” indicates drinks, “car” indicates cars, “building” indicates buildings, “aroma” indicates aromatics, and “cosme” indicates cosmetics. In the reference numeral 91, it is shown that the degree of similarity is larger between objects that correspond to white areas, and the degree of similarity is smaller between objects that correspond to black areas.

It is clear from the reference numeral 92 of the RSM 91 of FIG. 9 that the same category of commercial products has a higher degree of similarity, and expresses an ordinary classification in the brain. Furthermore, based on the reference numeral 93 of the RSM 91 of FIG. 9, some commercial products have similarity over categories. That is to say, they show a classification that is different from the ordinary category. Note that “category” refers to characters, drinks, cars, buildings, aromatics, and cosmetics.

Note that in the present specific example, a user may input an instruction to output a set of two or more objects having the degree of similarity that is a threshold or more. In such a case, the accepting unit 103 accepts the instruction.

Then, the object information acquisition unit 104 acquires, using the RSM stored in the brain information storage unit 102, one or more pairs of two or more objects having the degree of similarity that is a threshold or more. Note that, in such a case, the object information acquisition unit 104 does not use the diagonal elements of the RSM.

Then, the output unit 105 outputs the one or more pairs of two or more objects that were acquired by the object information acquisition unit 104. The output unit 105 outputs, for example, two pairs of object information (the character A and the car X) having the degree of similarity that is a threshold or more, or the like.

### Specific Example 3

Specific Example 3 is an example showing the result obtained from an experiment as will be described below using the brain information processing apparatus 1. Furthermore, brain activity information constituting brain information that is stored in the brain information storage unit 102 is information on the activation levels of parts of the brain when one subject is shown an object.

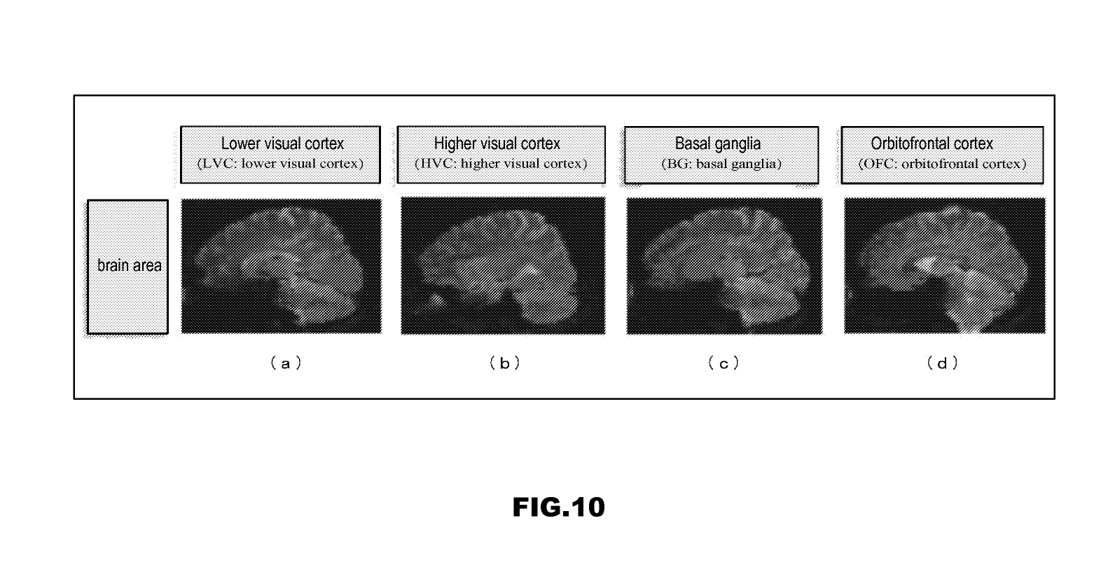

Then, a large number of images acquired by the fMRI device 5 when subjects are shown stimulus images are stored in the image storage unit 101. Examples of the images stored in the image storage unit 101 are shown in FIG. 10. In FIG. 10, (a) denotes an image in which the lower visual cortex, which is a partial area of the brain, is activated. Furthermore, (b) denotes an image in which the higher visual cortex, which is a partial area of the brain, is activated. Furthermore, (c) denotes an image in which the basal ganglia area is activated. Furthermore, (d) denotes an image in which the orbitofrontal cortex, which is a partial area of the brain, is activated.

Note that the lower visual cortex is an area that is activated when a feature of a local or simple image is detected. The feature of a local or simple image refers to an inclination or brightness of the outline of the image, or the like. Furthermore, the higher visual cortex is an area that is activated when a feature of a comprehensive or complicated image is detected. Detecting a feature of a comprehensive or complicated image is, for example, recognizing a physical object such as a face. Furthermore, the basal ganglia is an area that is activated when an unconscious compensation is obtained. “Unconscious compensation” refers to, for example, a physiological or monetary compensation or the like. The orbitofrontal cortex is an area that is activated when a subject feels a conscious joy or acquires the feeling of “like”. The case where a subject feels a conscious joy or acquires the feeling of “like” refers to the case where the subject feels luxuriousness, pleasant taste, or the like.

It is assumed that, in such a situation, a user has input an instruction to output brain activity information into the brain information processing apparatus 1. In response thereto, the accepting unit 103 accepts the instruction to output brain activity information.

Then, the feature vector acquisition unit 106 acquires images stored in the image storage unit 101.

Then, the feature vector acquisition unit 106 acquires the entire or parts of each image stored in the image storage unit 101. Then, the feature vector acquisition unit 106 acquires pixel values of the parts of the image. The pixel values of the parts of the image refer to the pixel values of images of the lower visual cortex, the higher visual cortex, the basal ganglia area, and the orbitofrontal cortex area of each image.

Then, the feature vector acquisition unit 106 acquires the baseline of the pixel values of pixels of the brain image. Note that the brain image that serves as a basis of the baseline is assumed to be held in advance by, for example, the feature vector acquisition unit 106.

Then, the feature vector acquisition unit 106 acquires two or more feature vectors that have, as elements, differences between the pixel values of parts of each image and the baseline pixel value. Note that the baseline pixel that serves as a basis of obtaining differences in pixel value is a pixel that is provided at a position corresponding to the pixel of the parts of the image.

Then, the brain activity information acquisition unit 107 acquires the degree of similarity between the two or more feature vectors.

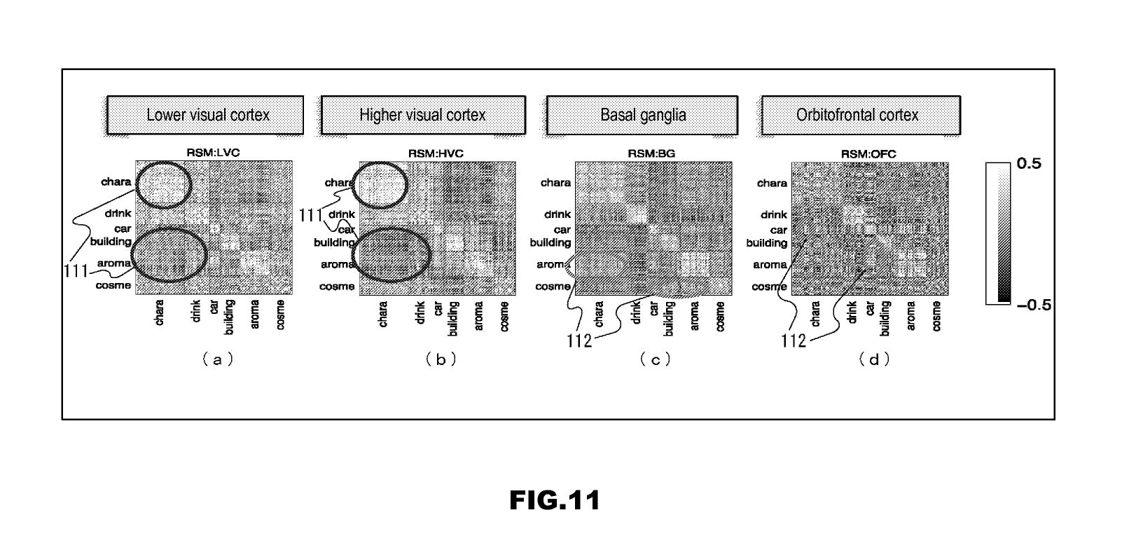

Then, the brain activity information acquisition unit 107 performs the above-described processing, using the degree of similarity between the two or more feature vectors, so as to acquire a brain expression similarity matrix (RSM), which is a symmetric matrix having degrees of similarity as elements. For example, the brain activity information acquisition unit 107 is assumed to have acquired the RSMs shown from (a) to (d) of FIG. 11. FIG. 11 shows the RSMs that correspond to the areas of the subject TH.

Then, the output unit 105 outputs the acquired RSMs of FIG. 11.

Note that it is clear from the reference numerals 111 of FIG. 11 that the higher visual cortex has a higher correlation within a category and a lower correlation between categories, as compared to the case of the lower visual cortex. This suggests that the higher visual cortex processes the commercial product category. Furthermore, it is clear from the reference numerals 112 of FIG. 11 that the basal ganglia and the orbitofrontal cortex that process an unconscious compensation or a conscious joy have a higher correlation between the categories. This indicates that similarity in the unconscious compensation or conscious joy over the categories is detectable.

According to the present specific example, visual features of the categories can be classified more clearly by the higher visual cortex. On the other hand, it is suggested that it is possible to detect the similarity in an unconscious compensation from the basal ganglia and the similarity in a conscious joy from the orbitofrontal cortex, over categories.

### Specific Example 4

Specific Example 4 is a specific example of the brain information processing apparatus 1. Specific Example 4 is an example showing the result obtained from an experiment as will be described below using the brain information processing apparatus 1. Furthermore, brain activity information constituting brain information constituting brain information that is stored in the brain information storage unit 102 is information on the activation levels in the basal ganglia area and the orbitofrontal cortex area, which are parts of the brain, when the four subjects are shown objects. That is, Specific Example 4 is an experiment result for use in determining whether or not an individual difference in both an unconscious compensation and a conscious joy exists between users. Note that the four subjects are the subject TH, the subject RA, the subject RH, and the subject PS.

It is here assumed that a large number of stimulus images are stored in a storing means (not shown). The stimulus images are the same as those in the foregoing specific examples.

Then, the stimulus images are displayed using a display screen or a projector, and are shown to the four subjects. Then, the brain activities of the four subjects when they are shown the stimulus images are measured using the fMRI device 5.

Then, it is assumed that a large number of images (for example, the reference numeral 71 of FIG. 7) acquired by the fMRI device 5 when the subjects are shown the stimulus images are stored in the image storage unit 101.

It is assumed that, in such a situation, a user has input an instruction to output brain activity information into the brain information processing apparatus 1. Then, the accepting unit 103 accepts the instruction to output brain activity information.

Then, the feature vector acquisition unit 106 acquires the images stored in the image storage unit 101. Then, the feature vector acquisition unit 106 acquires two or more pixel values of each image.

Then, the feature vector acquisition unit 106 acquires the baseline of the pixel values of pixels of the brain image. Note that the brain image that serves as a basis of the baseline is assumed to be held in advance by, for example, the feature vector acquisition unit 106.

Then, the vector acquisition unit 106 acquires, for each subject, two or more feature vectors that have differences between the pixel values of the basal ganglia area of each image and the baseline pixel value, as elements. Furthermore, the feature vector acquisition unit 106 acquire, for each subject, two or more feature vectors that have differences between the pixel values of the orbitofrontal cortex area of each image and the baseline pixel value, as elements. Note that the feature vector is generated for each image.

Then, the brain activity information acquisition unit 107 acquires, for each subject, the degree of similarity between the two or more feature vectors with respect to the basal ganglia area. Furthermore, the brain activity information acquisition unit 107 acquires, for each subject, the degree of similarity between the two or more feature vectors with respect to the orbitofrontal cortex area.

Then, the brain activity information acquisition unit 107 acquires, for each subject, a brain expression similarity matrix (RSM), which is a symmetric matrix that has degrees of similarity as elements, with respect to the basal ganglia area. Furthermore, the brain activity information acquisition unit 107 acquires, for each subject, a brain expression similarity matrix (RSM), which is a symmetric matrix that has degrees of similarity as elements, with respect to the orbitofrontal cortex area.

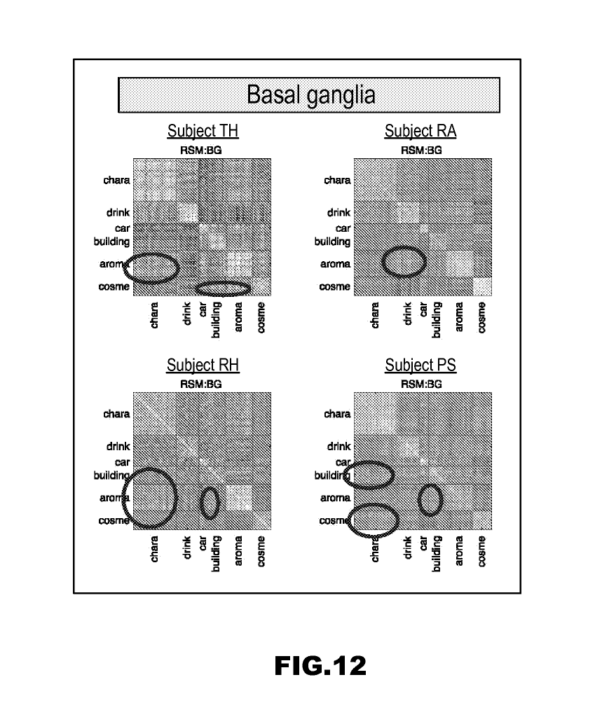

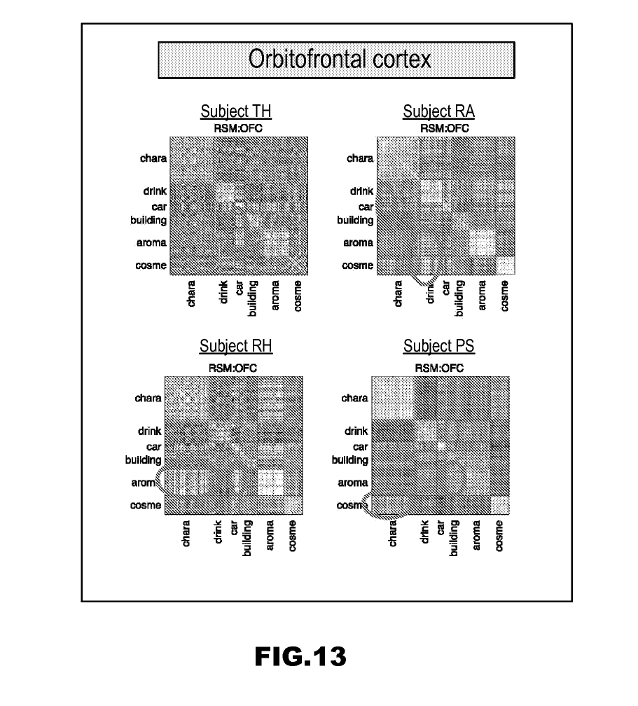

Then, the brain activity information acquisition unit 107 acquires, for each subject, the brain expression similarity matrices (RSM) shown in FIG. 12. The RSMs shown in FIG. 12 are RSMs that correspond to the basal ganglia area. Furthermore, the brain activity information acquisition unit 107 acquires, for each subject, the brain expression similarity matrices (RSM) shown in FIG. 13. The RSMs shown in FIG. 13 are RSMs that correspond to the orbitofrontal cortex area.

Then, the output unit 105 outputs the acquired RSMs (FIGS. 12 and 13).

As is clear from the regions surrounded by the ellipses of FIG. 12, the basal ganglia that processes an unconscious compensation includes an area that has a high correlation between categories and there are areas common to the subjects. As is clear from the regions surrounded by the ellipses of FIG. 13, the orbitofrontal cortex that processes a conscious joy as well includes areas that have a high correlation between categories and the patterns of the areas are different between the subjects.

Furthermore, in the present specific example, an analyzing means (not shown) may also perform analysis processing as will be described below. That is, the analyzing means generates, based on the RSM of each subject that was acquired by the brain activity information acquisition unit 107, a vector (RSM vector) in which elements of the RSM are aligned. Then, the analyzing means acquires information (X, Y) on the two-dimensional arrangement of the subject, using the MDS (Multi-dimensional scaling), which reduces the dimension number of the RSM vector while maintaining the distance relationship between the elements thereof.

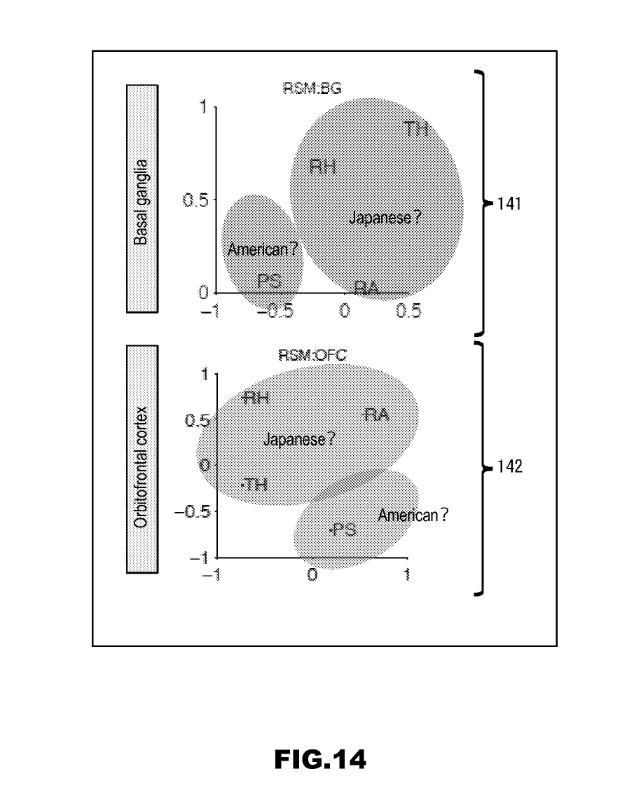

Then, the output unit 105 two-dimensionally outputs information on the subjects (TH, RA, RH, and PS) according to the information (X, Y) of arrangement of the subjects, which are results analyzed using the MDS. Such an output example is shown in FIG. 14. In FIG. 14, the reference numeral 141 denotes an output example of brain activity information obtained by analyzing the basal ganglia images of the subjects. Furthermore, the reference numeral 142 denotes an output example of the brain activity information obtained by analyzing the orbitofrontal cortex images of the subjects. According to the reference numerals 141 and 142, it is possible to understand attributive/cultural commonality and difference of the subjects although the number of the subjects is low. According to the reference numerals 141 and 142, it is also conceivable that there is a different characteristic between, for example, the Japanese and the Americans.

According to the present specific example, it is possible to map individual differences between subjects using an analysis method called MDS.

### Specific Example 5

In Specific Example 5, it is assumed that the brain information storage unit 102 has stored therein one or more pieces of brain information that includes feature vectors, which are brain activity information on brain activation levels when a subject is shown a large number of drink images, and object information, which is information on the object.

In such a situation, the same subject is shown another drink image. Then, the fMRI device 5 acquires an image indicating the brain activation levels at that time. Then, the feature vector acquisition unit 106 constructs a feature vector based on the image indicating the brain activation levels.

Then, the brain activity information acquisition unit 107 determines the feature vector that is most approximate to the feature vector acquired by the feature vector acquisition unit 106, among the feature vectors in the brain information storage unit 102.

Then, the brain activity information acquisition unit 107 acquires, from the brain information storage unit 102, the image that corresponds to the determined feature vector that is most approximate.

Then, the output unit 105 outputs the image. Note that this image is an image to which the subject feels the same sense as that of the other drink image shown to the subject. It is here preferable that the image of the other drink shown to the subject be output together with the image.

Thus, according to the present embodiment, it is possible to detect an object to which a person feels a similar sense using high level brain activity information indicating a latent consciousness.

Furthermore, according to the present embodiment, it is possible to detect, from a brain information database, an object with respect to which a person latently feels a similar sense to that of an object shown to her or him.

Furthermore, according to the present embodiment, it is possible to search for an object, taking into consideration individual characteristics.

Furthermore, it is possible to search an object using information on the brain activity of an appropriate part of the brain. Note that an appropriate part of the brain is, for example, the visual cortex, the basal ganglia, or the orbitofrontal cortex.

Furthermore, according to the present embodiment, it is possible to automatically construct a brain information database.

Note that the brain information processing apparatus 1 according to the present embodiment is available as a design evaluation system using high level brain activity information indicating a latent consciousness. That is, the brain information processing apparatus 1 may be a design evaluation apparatus.

Furthermore, the processing in the present embodiment may also be realized by software. Also, the software may be distributed by software downloading, or the like. Furthermore, the software may be recorded in a recording medium such as a CD-ROM and distributed. Note that the same applies to other embodiments in the present specification. Note that the software for realizing the brain information processing apparatus in the present embodiment is the following program. That is, this program has stored, in the recording medium, one or more pieces of brain information that include brain activity information, which is information on the activation level of a brain, when a subject is shown an object, and object information, which is information on the object. The program is a program for causing a computer to function as: an accepting unit that accepts brain activity information on a brain activation level when, in order that the one or more pieces of brain information to be stored in the recording medium are constructed, an object different from the object shown to the subject is shown to the same subject or a different subject; an object information acquisition unit configured to acquire, from the recording medium, one or more pieces of object information that are associated with one or more pieces of brain activity information that is approximate to the brain activity information accepted by the accepting unit to the extent of satisfying a predetermined condition; and an output unit configured to output the object information acquired by the object information acquisition unit.

Furthermore, the software that realizes the brain information processing apparatus of present embodiment has stored, for example, in the recording medium, two or more images that show brain activation levels, when a subject is shown two or more objects, and causes a computer to function as: an accepting unit configured to accept an instruction; a feature vector acquisition unit configured to acquire, in accordance with the instruction, two or more feature vectors that have, as elements, values relating to a change when compared with a baseline of pixel values of two or more pixels constituting the entire or a part of each of the two or more images; a brain activity information acquisition unit configured to acquire a degree of similarity between the two or more feature vectors, acquire a brain expression similarity matrix, which is a symmetric matrix having the degree of similarity as an element, and accumulate the brain expression similarity matrix in the brain information storage unit; and an output unit configured to output the brain expression similarity matrix.

Furthermore, the program is preferably a program configured such that the object information includes one or more pieces of metadata, which is attribute values of an object, and the object information acquisition unit includes: an object information determination part for determining two or more pieces of object information associated with two or more pieces of brain activity information that is approximate to brain activity information serving as a comparison target to the extent of satisfying a predetermined condition; and a static information acquisition part for performing statistical processing on the metadata of the two or more pieces of object information determined by the object information determination part and acquiring statistical information, and the output unit outputs the statistical information acquired by the static information acquisition part.

### Embodiment 2

The present embodiment will describe a brain information processing apparatus 2 that compares two or more pieces of brain activity information when a subject is shown two or more objects with teacher data in a DB, and outputs information on the object that is most approximate to the teacher data. Note that “teacher data” refers to brain information stored in a brain information storage unit 201, which will be described later, and is, for example, a feature vector extracted from the image of a bestselling commercial product.

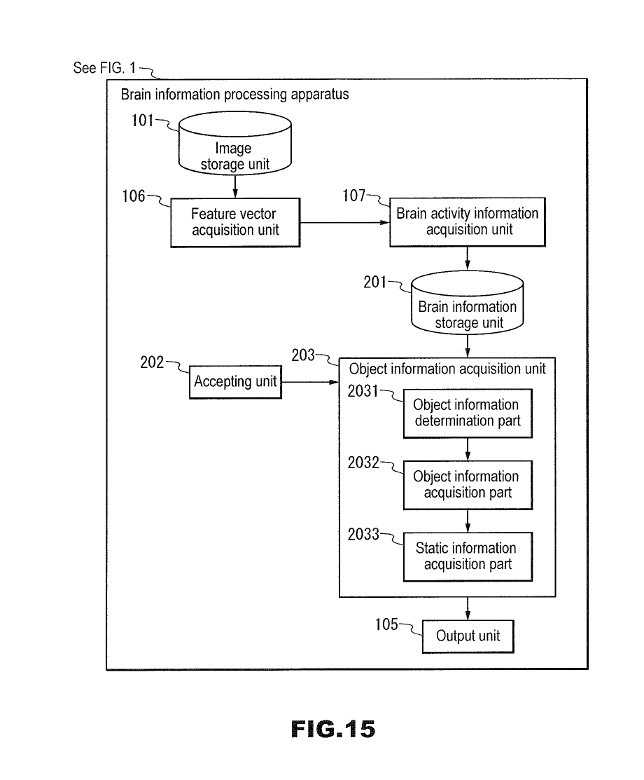

FIG. 15 is a block diagram showing the brain information processing apparatus 2 according to the present embodiment. The brain information processing apparatus 2 is provided with the image storage unit 101, a brain information storage unit 201, an accepting unit 202, an object information acquisition unit 203, the output unit 105, the feature vector acquisition unit 106, and the brain activity information acquisition unit 107. Note that the brain information processing apparatus 2 may also be provided with the above-described feature vector acquisition unit 106 and the brain activity information acquisition unit 107.

Furthermore, the object information acquisition unit 203 may also be provided with an object information determination part 2031, an object information acquisition part 2032, and a static information acquisition part 2033.

In the brain information storage unit 201, one piece of brain information may be stored. The brain information includes brain activity information. The brain information may include brain activity information and object information. Furthermore, the brain information may include only brain activity information. Furthermore, the brain information storage unit 201 preferably includes the brain information in association with personal information, which is personal information of the subject. Furthermore, here, the brain information is brain information that serves as a teacher, and is, for example, information including the brain activity information when the subject is shown a picture of a bestselling beer can.

The brain information storage unit 201 is preferably a nonvolatile recording medium, but is also realizable by a volatile recording medium. The process in which brain information is stored in the brain information storage unit 201 is not essential. For example, brain information may be stored in the brain information storage unit 201 via a recording medium, brain information transmitted via a communication line or the like may be stored in the brain information storage unit 201, or brain information that was input via an input device may be stored in the brain information storage unit 201.

The accepting unit 202 accepts two or more pieces of brain activity information, and two or more pieces of object information, which are pieces of information on two or more objects. The brain activity information is information on the brain activation level when, in order that one or more pieces of brain information to be stored in the brain information storage unit 102 are constructed, two or more objects that are different from the object shown to a subject are shown to the same or a different subject.

The accepting unit 202 may accept brain activity information and personal information that is associated with the brain activity information.

Any means such as a numerical keypad, a keyboard, a mouse, a menu screen, or the like may be used for inputting brain activity information and object information. The accepting unit 202 may be realized by a device driver for the input means such as a numerical keypad or a keyboard, control software for a menu screen, or the like.

The object information acquisition unit 203 acquires the degrees of similarity between brain activity information of the brain information stored in the brain information storage unit 201 and each of the two or more pieces of brain activity information accepted by the accepting unit 202, and acquires one or more pieces of object information associated with the brain activity information having a predetermined degree of similarity.

For example, a predetermined degree of similarity refers to the most approximate degree of similarity. In other words, it is also possible to compare the brain activity information of the brain information stored in the brain information storage unit 201 with each of the two or more pieces of brain activity information accepted by the accepting unit 202, to determine the brain activity information that is most approximate to the brain activity information of the brain information storage unit 201, and to acquire the object information associated with the brain activity information.

Furthermore, the predetermined degree of similarity may be sorting the object information in the descending order of the degree of similarity. That is, the object information acquisition unit 203 may acquire pieces of object information by sorting them in the descending order of the degree of similarity.

The object information acquisition unit 203 may acquire one or more pieces of brain activity information, from among the brain information associated with the personal information accepted by the accepting unit 202, acquires degrees of similarity between one or more pieces of brain activity information and the two or more pieces of brain activity information accepted by the accepting unit 202, and acquires the one or more pieces of object information associated with the brain activity information having the predetermined degree of similarity.

The object information acquisition unit 203 may ordinarily be realized by an MPU, a memory, or the like. The processing procedures of the object information acquisition unit 203 are ordinarily realized by software that is stored in a recording medium such as a ROM. However, the processing procedures may also be realized by hardware (dedicated circuits).

The object information determination part 2031 constituting the object information acquisition unit 203 determines one or two or more pieces of object information associated with one or two or more pieces of brain activity information that is approximate to the brain activity information serving as a comparison target to the extent of satisfying a predetermined condition. Note here that the brain activity information serving as a comparison target is the brain activity information stored in the brain information storage unit 201. The object information determination part 2031 determines, for example, N (N is 10, for example) pieces of object information associated with N pieces of brain activity information in the descending order of the degree of similarly to the brain activity information serving as a comparison target.

The object information acquisition part 2032 acquires a part or the entire of the object information determined by the object information determination part 2031. Here, the object information acquisition part 2032 acquires a part or the entire of the object information, from the pieces of object information accepted by the accepting unit 202.

The static information acquisition part 2033 performs statistical processing on metadata of the two or more pieces of object information determined by the object information determination part 2031, and acquires statistical information. The procedures of the static information acquisition part 2033 and the static information acquisition part 1043 are the same. Note that the object information acquisition unit 203 may not necessarily be provided with the static information acquisition part 2033.

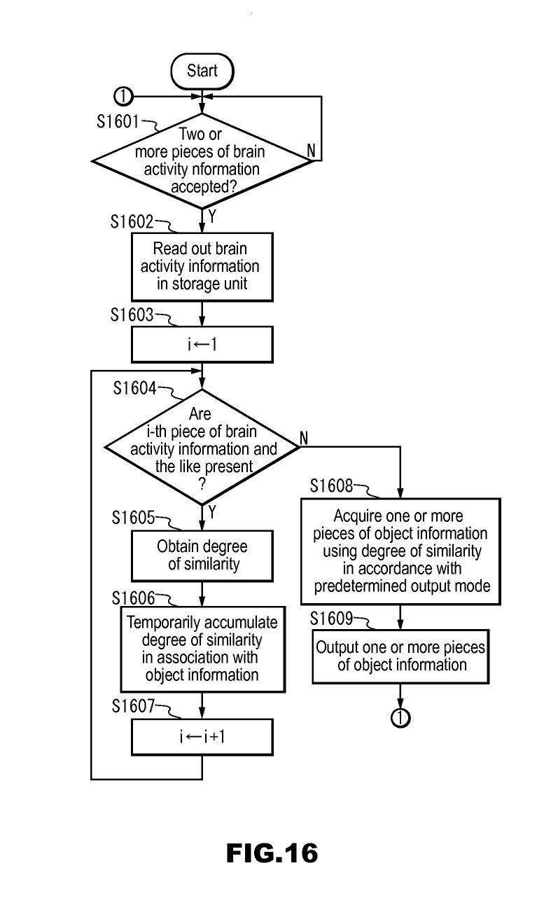

The following will describe the operation of the brain information processing apparatus 2 with reference to the flowchart of FIG. 16.

(Step S1601) The accepting unit 202 determines whether or not it has accepted two or more pieces of brain activity information and two or more pieces of object information, which are information on two or more objects. If the accepting unit 202 has accepted two or more pieces of brain activity information and the like, the procedure advances to step S1602, whereas if the accepting unit 202 has not accepted two or more pieces of brain activity information and the like, the procedure returns to step S1601.

(Step S1602) The object information acquisition unit 203 reads out the brain activity information stored in the brain information storage unit 201.

(Step S1603) The object information acquisition unit 203 substitutes 1 for a counter i.

(Step S1604) The object information acquisition unit 203 determines whether or not the i-th piece of brain activity information and the like are present in the information accepted in step S1601. If the i-th piece of brain activity information and the like are present, the procedure advances to step S1605, whereas if the i-th piece of brain activity information and the like are not present, the procedure advances to step S1608.

(Step S1605) The object information acquisition unit 203 calculates the degree of similarity between the brain activity information read out in step S1602 and the i-th piece of brain activity information.

(Step S1606) The object information acquisition unit 203 temporarily accumulates, in a recording medium (not shown), the degree of similarity calculated in step S1605 in association with the i-th piece of object information.

(Step S1607) The object information acquisition unit 203 increments the counter i by 1. The procedure returns to step S1604.

(Step S1608) The object information acquisition unit 203 acquires one or more pieces of object information using the degree of similarity accumulated in the recording medium (not shown) in accordance with a predetermined output mode.

(Step S1609) The output unit 105 outputs the one or more pieces of object information acquired in step S1608. The procedure returns to step S1601.

Note that in the flowchart of FIG. 16, the processing ends due to power off or an interruption at the end of the processing.

The following will describe the specific operation of the brain information processing apparatus 2 according to the present embodiment. A specific example of the apparatus for acquiring an image indicating the brain activation level when a subject is shown an object is the fMRI device 5 described in Embodiment 1.

### Specific Example 1

Specific Example 1 shows an experiment result when the subject RA was shown stimulus images that are images of bestselling beer cans.

In Specific example 1, the fMRI device 5 is used to measure the brain activity of the subject RA when he or she is shown the stimulus images. That is, images acquired by the fMRI device 5 when the subject RA is shown the stimulus images are stored in the image storage unit 101. Note here that the images are fMRI brain activity patterns.

Then, the feature vector acquisition unit 106 acquires two or more pixel values of the basal ganglia area of the images stored in the image storage unit 101.

Then, the feature vector acquisition unit 106 acquires the baseline of the pixel values of pixels of the basal ganglia area of the brain image. Note that the brain image that serves as a basis of the baseline is assumed to be held in advance by, for example, the feature vector acquisition unit 106.

Then, the feature vector acquisition unit 106 acquires a feature vector that has, as elements, differences between the pixel values of the image and the baseline pixel value. The feature vector is a vector showing the operation of the basal ganglia area of the subject RA when he or she is shown the bestselling beer can.

Then, the feature vector acquisition unit 106 acquires two or more pixel values of a higher visual cortex of the images stored in the image storage unit 101. Then, the feature vector acquisition unit 106 acquires the baseline of the pixel values of pixels of the higher visual cortex of the brain image.

Then, the feature vector acquisition unit 106 acquires a feature vector that has, as elements, differences between the pixel values of the image and the baseline pixel value. The feature vector is a vector showing the operation of the higher visual cortex of the subject RA when he or she is shown the bestselling beer can.

Similarly, the subject RA was shown images of a large number of other drink cans, and fMRI brain activity patterns at that time are acquired by the fMRI device 5 and accumulated in a recording medium (not shown). Note that the fMRI brain activity pattern is an image from which the brain activity is recognized.

Then, similarly to the foregoing description, the feature vector acquisition unit 106 acquires, for each image of the recording medium (not shown), a feature vector showing the operation of the basal ganglia area and a feature vector showing the operation of the higher visual cortex, and temporarily accumulates, in the recording medium (not shown), the feature vectors in association with the images of the drink cans.

Then, the accepting unit 202 accepts, from the recording medium (not shown), two or more feature vectors showing the operation of the basal ganglia area, and object information that correspond to the respective feature vector. In this context, the object information refers to, for example, a drink can image, a drink name, and a sales company.

Then, the object information acquisition unit 203 compares the feature vectors corresponding to the basal ganglia area that were accepted by the accepting unit 202 with the feature vectors corresponding to the basal ganglia area of the subject RA when he or she is shown the bestselling beer cans, calculates the degree of similarity for each feature vector accepted by the accepting unit 202, and accumulates the degrees of similarity in association with the object information.

Then, the object information acquisition unit 203 sorts the pieces of object information in the descending order of the degree of similarity.

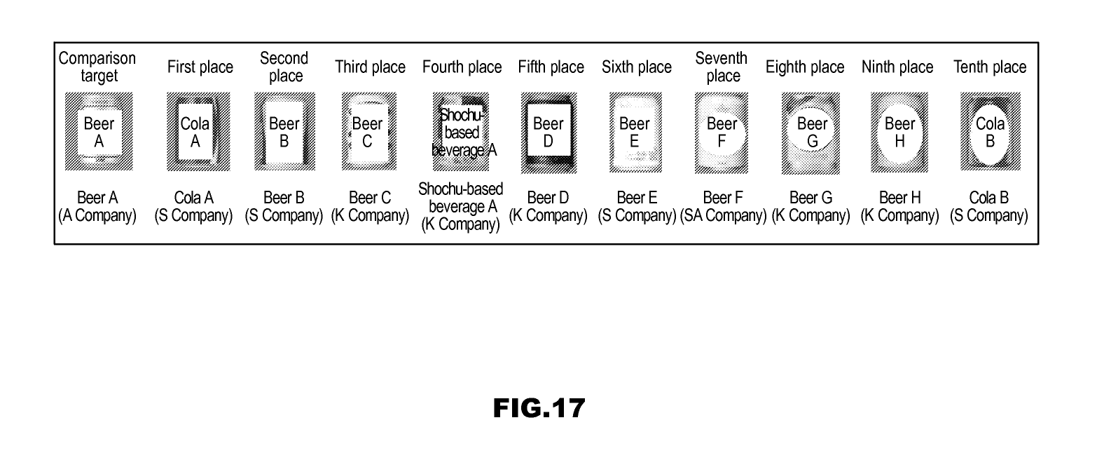

Then, the output unit 105 outputs two or more pieces of object information sorted in order of the degree of similarity. Note here that the output unit 105 is assumed to also output the images of the bestselling beer cans. Furthermore, an output example in such a case is shown in FIG. 17. FIG. 17 shows the degrees of similarity in the basal ganglia of the subject RA. Furthermore, the image serving as the comparison target of FIG. 17 is the image of the bestselling beer can.

In view of the brain activity in the basal ganglia with respect to “Super Dry”, which is a bestselling beer, it is recognizable that the brain activity has a high degree of similarity to the brain activity in the case of the well-selling commercial product in each category (such as a carbonated drink or premium beer), and an unconscious compensation of the design indicates sales trend.

Then, the accepting unit 202 accepts, from the recording medium (not shown), two or more feature vectors showing the operation of the higher visual cortex, and object information that correspond to the respective feature vectors. In this context, the object information refers to, for example, a drink can image, a drink name, and a sales company.

Then, the object information acquisition unit 203 compares the feature vectors corresponding to the higher visual cortex that were accepted by the accepting unit 202 with the feature vectors corresponding to the higher visual cortex of the subject RA when he or she is shown the bestselling beer can, calculates the degree of similarity for each feature vector accepted by the accepting unit 202, and accumulates the degree of similarity in association with the object information.

Then, the object information acquisition unit 203 sorts the pieces of object information in the descending order of the degree of similarity.

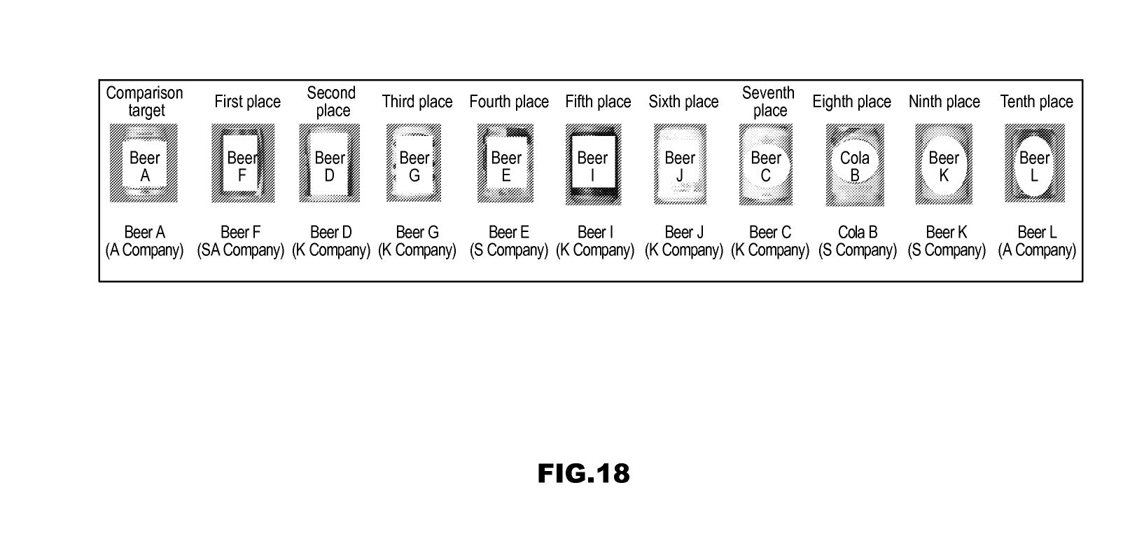

Then, the output unit 105 outputs two or more pieces of object information sorted in order of the degree of similarity. Note here that the output unit 105 is assumed to also output the images of the bestselling beer cans. Furthermore, an output example in such a case is shown in FIG. 18. FIG. 18 shows the degrees of similarity in the higher visual cortex of the subject RA. Furthermore, the image serving as a comparison target of FIG. 18 is the image of the bestselling beer can.

### Specific Example 2

In Specific Example 2, the subject RH was shown expensive skin care cosmetic posters (stimulus images). Then, the brain activity of the subject RA when he or she was shown the stimulus images was measured using the fMRI device 5.

Then, the images acquired by the fMRI device 5 when the subject RH was shown the stimulus images were stored in the image storage unit 101. Note here that the images are fMRI brain activity patterns.

Then, the feature vector acquisition unit 106 acquires two or more pixel values in the orbitofrontal cortex area of the images stored in the image storage unit 101. Then, the feature vector acquisition unit 106 acquires the baseline of the pixel values of pixels of the orbitofrontal cortex area of the brain image. Note that the brain image that serves as a basis of the baseline is assumed to be held in advance by, for example, the feature vector acquisition unit 106.

Then, the feature vector acquisition unit 106 acquires feature vectors that have, as elements, differences between the pixel values of the image and the baseline pixel value. This feature vector is a vector showing the operation of the orbitofrontal cortex area of the subject RA when he or she was shown the expensive skin care cosmetic poster.

Similarly, the subject RH was shown a large number of other cosmetics posters, and fMRI brain activity patterns at that time are acquired by the fMRI device 5 and accumulated in the image storage unit 101.

Then, similarly to the foregoing description, the feature vector acquisition unit 106 acquires, for each image of the image storage unit 101, a feature vector showing the operation of the orbitofrontal cortex area, and temporarily accumulates the feature vectors in association with the drink can images in the recording medium (not shown).

Then, the object information acquisition unit 203 compares the feature vectors corresponding to the orbitofrontal cortex area that were accepted by the accepting unit 202 with the feature vectors corresponding to the orbitofrontal cortex area of the subject RH when he or she is shown the expensive skin care cosmetic poster, calculates the degree of similarity for each feature vector accepted by the accepting unit 202, and accumulates the degrees of similarity in association with the object information.

Then, the object information acquisition unit 203 sorts the pieces of object information in the descending order of the degree of similarity.

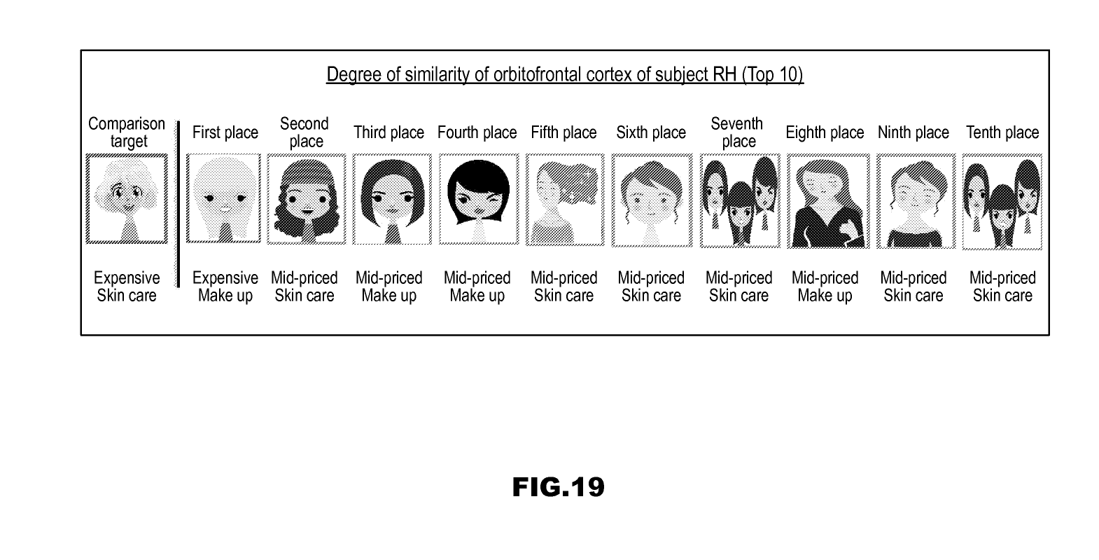

Then, the output unit 105 outputs two or more pieces of object information sorted in order of the degree of similarity. Note here that the output unit 105 is assumed to also output the expensive skin care cosmetic poster. Furthermore, an output example in such a case is shown in FIG. 19. FIG. 19 shows the degrees of similarity in the orbitofrontal cortex of the subject RH. Furthermore, the image serving as a comparison target of FIG. 19 is the image of the expensive skin care cosmetic poster.

In FIG. 19, in the orbitofrontal cortex, the activity for the expensive skin care poster is similar to that for an expensive makeup poster. This suggests that the orbitofrontal cortex processes luxuriousness. Furthermore, in FIG. 19, posters of mid-priced commercial products are lined-up from the second place onwards, but the influence on the brain may be different between the cases of the higher place and the lower place. Therefore, comparison is possible between the brand image that is considered by the own company and the brand image accepted by users. Note that the influence on the brain is considered here as luxuriousness.

According to the present embodiment, it is possible to detect an object with respect to which a person latently feels the most similar sense to that of given teacher data when the person is shown two or more objects.

Furthermore, the brain information processing apparatus 2 of the present embodiment is available for evaluation of a design. The brain information processing apparatus 2 may be a design evaluation apparatus.

Note that software that realizes the brain information processing apparatus of present embodiment is the following program. That is, this program is a program that has stored, for example, in the recording medium, two or more images that show brain activation levels, when a subject is shown two or more objects, and causes a computer to function as: an accepting unit configured to accept an instruction; a feature vector acquisition unit configured to acquire, in accordance with the instruction, two or more feature vectors that have, as elements, values relating to a change when compared with a baseline of pixel values of two or more pixels constituting the entire or a part of each of the two or more images; a brain activity information acquisition unit configured to acquire a degree of similarity between the two or more feature vectors, acquire a brain expression similarity matrix, which is a symmetric matrix having the degree of similarity as an element, and accumulate the brain expression similarity matrix in the brain information storage unit; and an output unit configured to output the brain expression similarity matrix.

Furthermore, the program is preferably a program configured such that the object information includes one or more pieces of metadata, which is attribute values of an object, and the object information acquisition unit includes: an object information determination part for determining two or more pieces of object information associated with two or more pieces of brain activity information that is approximate to brain activity information serving as a comparison target to the extent of satisfying a predetermined condition; and a static information acquisition part for performing statistical processing on the metadata of the two or more pieces of object information determined by the object information determination part and acquiring statistical information, and the output unit outputs the statistical information acquired by the static information acquisition part.

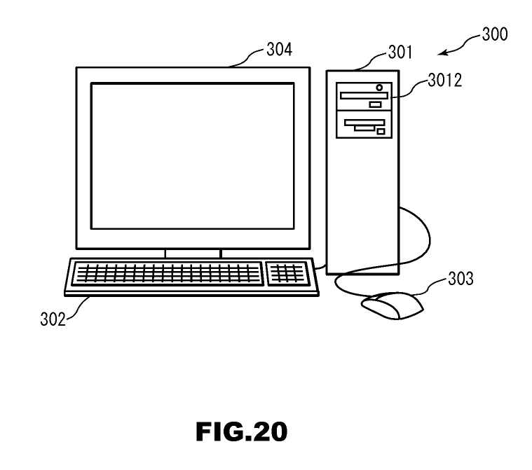

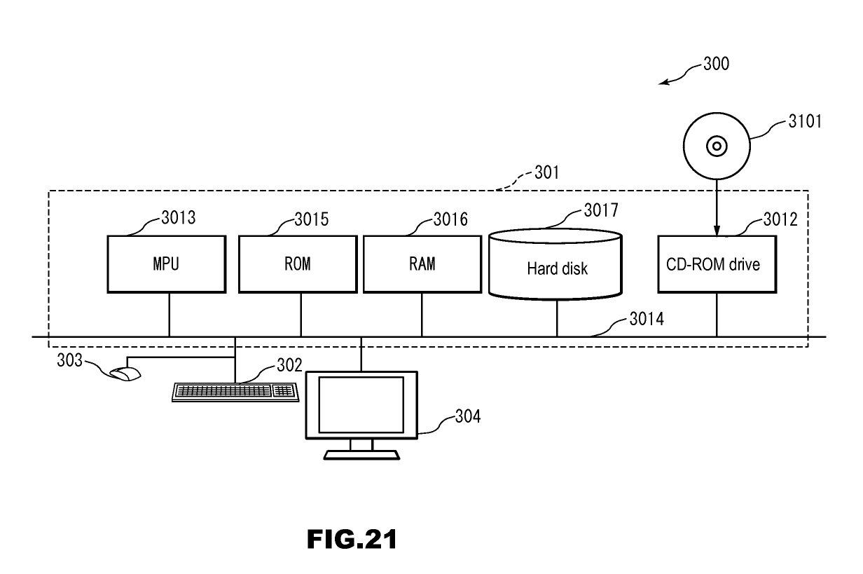

Furthermore, FIG. 20 is an overview of a computer that executes the program described in the present specification to realize the brain information processing apparatuses according to the above-described various types of embodiments. The foregoing embodiments can be realized by computer hardware and a computer program that is executed thereon. FIG. 20 is an overview diagram showing a computer system 300, and FIG. 21 is a block diagram showing the system 300.

In FIG. 20, the computer system 300 includes a computer 301 with a CD-ROM drive, a keyboard 302, a mouse 303, and a monitor 304.

In FIG. 21, the computer 301 includes, in addition to a CD-ROM drive 3012, an MPU 3013, a bus 3014, a ROM 3015, a RAM 3016, and a hard disk 3017. Note that the bus 3014 is connected to the MPU 3013 and the CD-ROM drive 3012. Furthermore, the ROM 3015 has stored therein a program such as a boot-up program. Furthermore, the RAM 3016 is connected to the MPU 3013, and configured to temporarily store a command of an application program and provide a temporary storage area. Furthermore, the hard disk 3017 is configured to store an application program, a system program, and data. The computer 301 may further include a network card for providing connection to the LAN, although it is not shown here.

The program that causes the computer system 300 to execute the functions of the brain information processing apparatuses of the foregoing embodiments is stored in the CD-ROM 3101, and the CD-ROM 3101 may be inserted into the CD-ROM drive 3012 and be further transmitted to the hard disk 3017. Alternatively, the program may be transmitted to the computer 301 via a network (not shown) and may be stored in the hard disk 3017. The program is loaded onto the RAM 3016 at the time of execution. The program may also be loaded directly from the CD-ROM 3101 or the network.

The program may not necessarily include an operating system, a third party program, or the like that causes the computer 301 to execute the functions of the brain information processing apparatuses of the foregoing embodiments. The program may only need to include the command part that calls an appropriate function (module) in a controlled aspect so as to obtain a desired result. The operation of the computer system 300 is well known, and thus a detailed description thereof is omitted.

Furthermore, a single or multiple computers that execute the program may be provided. That is, centralized processing may be performed or decentralized processing may be performed.

Furthermore, in the foregoing embodiments, each process (each function) may be realized by a single apparatus (system) performing centralized processing, or by multiple apparatuses performing decentralized processing.

The present invention is not limited to the foregoing embodiments, and various modifications are possible and are of course encompassed in the scope of the present invention.

### INDUSTRIAL APPLICABILITY

As described above, the brain information processing apparatus according to the present invention has an effect of capable of detecting an object to which a person feels a similar sense using high-level brain activity information indicating a latent consciousness, and is advantageous as a brain information processing apparatus or the like.

### LIST OF REFERENCE NUMERALS

1, 2 Brain information processing apparatus
5 fMRI device
101 Image storage unit
102, 201 Brain information storage unit
103, 202 Accepting unit
104, 203 Object information acquisition unit
105 Output unit
106 Feature vector acquisition unit
107 Brain activity information acquisition unit

1-17. (canceled)

18. A brain information processing apparatus comprising:
a brain information storage unit in which one or more pieces of brain information can be stored, the one or more pieces of brain information including brain activity information, which is information on a brain activation level, when a subject is shown an object, and object information, which is information on the object, the one or more pieces of brain information being information on an activation level of a predetermined partial area of a brain, the brain information processing apparatus further comprising:
an accepting unit configured to accept brain activity information on a brain activation level when, in order that the one or more pieces of brain information to be stored in the brain information storage unit are constructed, an object different from the object shown to the subject is shown to the same subject or a different subject;
an object information acquisition unit configured to acquire, from the brain information storage unit, one or more pieces of object information associated with one or more pieces of brain activity information that is approximate to the brain activity information accepted by the accepting unit to the extent of satisfying a predetermined condition, with respect to the partial area; and
an output unit configured to output the object information acquired by the object information acquisition unit.

19. The brain information processing apparatus according to claim 18,
wherein the accepting unit accepts two or more pieces of brain activity information on a brain activation level when, in order that the one or more pieces of brain information to be stored in the brain information storage unit are constructed, two or more objects different from the object shown to the subject are shown to the same subject or a different subject and two or more pieces of object information, which is information on the two or more objects, and
the object information acquisition unit acquires a degree of similarity between the brain activity information of the brain information stored in the brain information storage unit and the two or more pieces of brain activity information accepted by the accepting unit, so as to acquire object information that is associated with the brain activity information having a predetermined degree of similarity.

20. The brain information processing apparatus according to claim 18,
wherein the brain information storage unit includes the brain information in association with personal information, which is information on an individual subject,
the accepting unit accepts brain activity information and personal information associated with the brain activity information, and
the object information acquisition unit acquires, from the brain information associated with the personal information accepted by the accepting unit, one or more pieces of brain activity information, so as to acquire one or more pieces of object information that are associated with the one or more pieces of brain activity information.

21. The brain information processing apparatus according to claim 19,
wherein the brain information storage unit includes the brain information in association with personal information, which is information on an individual subject,
the accepting unit accepts brain activity information and personal information associated with the brain activity information, and
the object information acquisition unit acquires, from the brain information associated with the personal information accepted by the accepting unit, one or more pieces of brain activity information, so as to acquire one or more pieces of object information that are associated with the one or more pieces of brain activity information.

22. The brain information processing apparatus according to claim 18,
wherein the object information includes one or more pieces of metadata, which is an attribute value of an object,
the object information acquisition unit includes:
an object information determination part for determining two or more pieces of object information associated with two or more pieces of brain activity information that is approximate to brain activity information serving as a comparison target to the extent of satisfying a predetermined condition; and
a static information acquisition part for performing statistical processing on the metadata of the two or more pieces of object information determined by the object information determination part and acquiring statistical information, and
the output unit outputs the statistical information acquired by the static information acquisition part.

23. The brain information processing apparatus according to claim 19,
wherein the object information includes one or more pieces of metadata, which is an attribute value of an object,
the object information acquisition unit includes:
an object information determination part for determining two or more pieces of object information associated with two or more pieces of brain activity information that is approximate to brain activity information serving as a comparison target to the extent of satisfying a predetermined condition; and
a static information acquisition part for performing statistical processing on the metadata of the two or more pieces of object information determined by the object information determination part and acquiring statistical information, and
the output unit outputs the statistical information acquired by the static information acquisition part.

24. The brain information processing apparatus according to claim 18,
wherein the partial area of the brain is a brain area that includes a visual cortex of the brain.

25. The brain information processing apparatus according to claim 18,
wherein the partial area of the brain is a brain area that includes the basal ganglia.

26. The brain information processing apparatus according to claim 18,
wherein the partial area of the brain is a brain area that includes the orbitofrontal cortex.

27. The brain information processing apparatus according to claim 18, further comprising:
an image storage unit in which two or more images that show brain activation levels when a subject is shown two or more objects can be stored;
a feature vector acquisition unit configured to acquire two or more feature vectors that have, as elements, values relating to a change as compared with a baseline of pixel values of two or more pixels constituting each of the two or more images; and
a brain activity information acquisition unit configured to acquire a degree of similarity between the two or more feature vectors, acquire a brain expression similarity matrix, which is a symmetric matrix having the degree of similarity as an element, and accumulate the brain expression similarity matrix in the brain information storage unit.

28. The brain information processing apparatus according to claim 19, further comprising:
an image storage unit in which two or more images that show brain activation levels when a subject is shown two or more objects can be stored;
a feature vector acquisition unit configured to acquire two or more feature vectors that have, as elements, values relating to a change as compared with a baseline of pixel values of two or more pixels constituting each of the two or more images; and
a brain activity information acquisition unit configured to acquire a degree of similarity between the two or more feature vectors, acquire a brain expression similarity matrix, which is a symmetric matrix having the degree of similarity as an element, and accumulate the brain expression similarity matrix in the brain information storage unit.

29. A brain information processing apparatus comprising:
an image storage unit in which two or more images that show brain activation levels when a subject is shown two or more objects can be stored;
an accepting unit configured to accept an instruction;
a feature vector acquisition unit configured to acquire, in accordance with the instruction, two or more feature vectors that have, as elements, values relating to a change as compared with a baseline of pixel values of two or more pixels constituting the entire or a part of each of the two or more images;
a brain activity information acquisition unit configured to acquire a degree of similarity between the two or more feature vectors, acquire a brain expression similarity matrix, which is a symmetric matrix having the degree of similarity as an element, and accumulate the brain expression similarity matrix in a brain information storage unit; and
an output unit configured to output the brain expression similarity matrix.

30. A brain information processing method in which a recording medium has stored therein one or more pieces of brain information that includes brain activity information, which is information on an activation level of a brain, when a subject is shown an object, and object information, which is information on the object, the brain activity information being information from which an activation level of a predetermined partial area of the brain can be extracted, the method being realized by an accepting unit, an object information acquisition unit, and an output unit, and comprising:
an accepting step of the accepting unit accepting brain activity information on a brain activation level when, in order that the one or more pieces of brain information to be stored in the recording medium are constructed, an object different from the object shown to the subject is shown to the same subject or a different subject;
an object information acquiring step of the object information acquisition unit acquiring, from the recording medium, one or more pieces of object information associated with one or more pieces of brain activity information that is approximate to the brain activity information accepted in the accepting step to the extent of satisfying a predetermined condition, with respect to the partial area; and
an outputting step of the output unit outputting the object information acquired in the object information acquiring step.

31. The brain information processing method according to claim 30,
wherein in the accepting step, two or more pieces of brain activity information on a brain activation level when, in order that the one or more pieces of brain information to be stored in the recording medium are constructed, two or more objects different from the object shown to the subject are shown to the same subject or a different subject and two or more pieces of object information, which is information on the two or more objects, are accepted, and
in the object information acquiring step, a degree of similarity between the brain activity information of the brain information stored in the recording medium and the two or more pieces of brain activity information accepted in the accepting step is acquired so as to acquire object information that is associated with the brain activity information having a predetermined degree of similarity.

32. A brain information processing method in which a recording medium has stored therein two or more images that show brain activation levels when a subject is shown two or more objects, the method being realized by a feature vector acquisition unit, a brain activity information acquisition unit, and an output unit, and comprising:
a feature vector acquiring step of the feature vector acquisition unit acquiring two or more feature vectors that have, as elements, values relating to a change as compared with a baseline of pixel values of two or more pixels constituting the entire or a part of each of the two or more images;
a brain activity information acquiring step of the brain activity information acquisition unit acquiring a degree of similarity between the two or more feature vectors, and acquiring a brain expression similarity matrix, which is a symmetric matrix having the degree of similarity as an element; and
an outputting step of the output unit outputting the brain expression similarity matrix.

33. The brain information processing apparatus according to claim 18,
wherein the brain activity information is information from which an activation level of a specific partial area of predetermined multiple partial areas of the brain can be extracted, and
the object information acquisition unit acquires, from the brain information storage unit, one or more pieces of object information associated with one or more pieces of brain activity information that is approximate to the extent of satisfying a predetermined condition with respect to each specific partial area of the predetermined multiple partial areas.
